(19) United States
(12) Patent Application Publication
(10) Pub. No.: US 2001/0041670 A1
(43) Pub. Date: Nov. 15, 2001

(54) Thrombospondin-binding region of histidine-rich glycoprotein and method of use

(76) Inventors: Ronit Simantov, Scarsdale, NY (US); Roy L. Silverstein, New York, NY (US)

Correspondence Address: Irving Feit, Esq., HOFFMANN & BARON, LLP, 6900 Jericho Turnpike, Syosset, NY, 11791, US

(21) Appl. No.: 09/730,379

(22) Filed: Dec. 5, 2000

Related U.S. Application Data
(60) Provisional application No. 60/169,205, filed on Dec. 6, 1999.

(51) Int. Cl. A61K 38/16; A61K 31/70; A61K 39/395

(52) U.S. Cl. 514/8; 514/44; 435/7.23; 424/155.1

(57) ABSTRACT

Thrombospondin-binding molecules and fragments comprising one or more regions of the TSP-1-binding domains of Histidine-Rich Glycoprotein (HRGP) are provided. Also provided are homologs of TSP-1-binding domains of Histidine-Rich Glycoprotein. Therapeutic methods of use of these thrombospondin-binding molecules and fragments, as well as anti-HRGP antibodies and antibody fragments are disclosed. These methods are useful in modulating a variety of physiological and pathological processes including angiogenesis, inflammatory responses, embryogenesis and tumor proliferation. A range of assay methods for diagnosis and for detection, quantitation and identification of modulators of the TSP-l-binding activity of HRGP are also provided. In addition screening assays for compounds that specifically modulate HRGP expression are provided. These assays are useful in a range of formats from individual assays to library screening as well as high throughput assays. Compounds identified by such methods are useful as therapeutic candidate molecules or as lead compounds in the development of therapeutic molecules.

### RELATED APPLICATION

This application claims the benefit of the Provisional Patent Application U.S. Ser. No. 60/169,205 filed on Dec. 6, 1999 and entitled “Thrombospondin-Binding Region of Histidine-Rich Glycoprotein and Methods of Use.”

This work was supported in part by a Clinical Associate Physician Award M01RR00047 from the General Clinical Research Centers of the National Institutes of Health and research grant awards R01HL42540 and R29HL58559 from the National Institutes of Health. The government may have certain rights to this invention.

### FIELD OF THE INVENTION

This invention relates to the field of regulation of growth and proliferation, such as in the accretion of new blood vessels (angiogenesis), particularly for treatment of cardiovascular disease. The invention also relates to the reduction of angiogenesis, including growth suppression and arrest, and apoptosis in normal development, for example in embryogenesis, and in a wide range of disorders and diseases, including those involving tumors, malignancies, neoplastic and other pathological conditions and homeostatic imbalances in the control of growth and development.

### BACKGROUND

Angiogenesis, the development of new blood vessels, is necessary for a variety of normal physiologic processes as well as for the growth and proliferation of tumors. The identification of natural modulators of angiogenesis is essential to the understanding of this complex process and provides attractive targets for therapeutic intervention. Histidine-rich glycoprotein (HRGP) is a plasma protein with an unusually high histidine and proline content that circulates in relatively high concentrations (1.5 μM), but has no known function in vivo (1). The entire 525 amino acid sequence of human HRGP is disclosed in GenBank under accession number P04196 and is hereby incorporated by reference in its entirety.

Several in vitro interactions of HRGP have been described, including binding to metals, heparin and several proteins including plasminogen, fibrinogen and thrombospondin-1 (TSP-1) (2, 3). TSP-1 interaction with the membrane protein CD36 is known to play a role in platelet-tumor and platelet-monocyte adhesion, angiogenesis, and in monocyte uptake of apoptotic cells. Surface bound HRGP can accelerate the activation of plasminogen by tissue plasminogen activator (tPA), suggesting a role for HRGP in the fibrinolytic system (4). Although there are reports of inherited HRGP polymorphisms and elevated HRGP levels in families with thromboembolic disease, the role of HRGP in thrombosis and fibrinolysis in humans has not been determined (5).

HRGP binds with high affinity to TSP-1 (6), a multifunctional 450 kDa homotrimeric adhesive glycoprotein that is a potent inhibitor of angiogenesis (7-9). TSP-1 is secreted by activated platelets and a variety of normal vascular cells including endothelial and smooth muscle cells (10), and has been shown to inhibit endothelial cell (EC) proliferation, migration, and tube formation in response to multiple angiogenic stimuli (11). The anti-angiogenic activity of TSP-1 has been localized to the properidin-like type I repeats, and synthetic peptides derived from the type I domains have been found to have potent anti-angiogenic activity in vivo and in assays of EC function (8).

Although TSP-1 interacts with a number of distinct cellular receptors, CD36 has been recognized as the critical anti-angiogenesis receptor for TSP-1 (12, 13). The binding of TSP-1 to CD36 is mediated by the peptide sequence cysteine-serine-valine-threonine-cysteine-glycine (CSVTCG), the same type I repeat shown to have anti-angiogenic activity. (14, 15).

There is a need for new and specific methods that promote angiogenesis to intervene therapeutically in conditions, disorders and diseases where the blood supply to a tissue is reduced. Cardiovascular disease is an example of such a disease where the stimulation of angiogenesis is therapeutic. Stimulation of growth of new blood vessels in heart muscle after a heart attack would be one application of an angiogenic method.

Similarly, there is a need for novel and targeted methods to reduce angiogenesis in conditions, disorders and diseases where there is an over-abundant blood supply. Growth of tumors and malignancies could be reduced or blocked by such methods that depend on an anti-angiogenic activity.

### SUMMARY OF THE INVENTION

The present invention relates to proteins comprising the thrombospondin (TSP)-binding motif of the Histidine-rich glycoprotein (HRGP) from natural, synthetic or recombinant sources. The molecular weight of these proteins may vary from about 7 Kda up to about 60 Kda, or alternatively, the proteins may have a molecular weight between about 70 Kda and 200 Kda. Clinical grade preparations of the proteins of the present invention, preferably prepared under GMP conditions are useful as pharmaceutical compounds when delivered in suitable carriers.

Unexpectedly, it has been found that the activity of thrombospondin (TSP-1), an anti-angiogenic protein, can be modulated by stimulating or inhibiting the activity of HRGP. Alternatively, the anti-angiogenic activity of thrombospondin (TSP-1) can be modulated by stimulating or inhibiting the expression of HRGP.

The present invention provides methods of regulating the thrombospondin activity in a cell by modulating thrombospondin (TSP-l)-binding activity in the cell. The modulation of TSP-1-binding activity includes stimulation, i.e. increasing TSP-1-binding activity. The modulation of TSP-1-binding activity also includes inhibition, i.e. reducing TSP-1-binding activity.

The methods of the present invention for modulating the TSP-1-binding activity include methods for modulating the activity of histidine-rich glycoprotein (HRGP). HRGP may be modulated, for example, either by reducing or by increasing its expression. Alternatively, HRGP may be modulated, for example by reducing or by increasing its TSP-1 binding activity. This modulation of TSP-1-binding activity may be in a cell free system, such as in a cell free sample for an assay. The modulation of TSP-1-binding activity may also be in a cell or a tissue, or in a whole mammal. The mammal in which the TSP-1-binding activity is modulated may be any mammal, such as for instance a primate, particularly a human.

In one embodiment, the present invention provides a method of inhibiting angiogenesis in a tissue by inhibiting the expression of HRGP, or by inhibiting the activity of HRGP. This method may be applied in the treatment of conditions, disorders or diseases that involve enhanced or unregulated angiogenesis. An example of a disorder or disease treatable by the methods of the present invention is a cancer that gives rise to a tumor, particularly a malignant tumor undergoing angiogenesis.

Other cancers treatable by the methods of the present invention include anal cancer, bladder cancer, small cell lung cancer, non-small cell lung cancer, bone cancer, brain cancer, breast cancer, cervical cancer, chondrosarcoma, clear cell adenosarcoma (DES), colorectal cancer, endometrial cancer, esophageal cancer, cancer of the eye, cancer of the eyelid, kaposi's sarcoma, kidney cancer, cancer of the larynx, leiomyosarcoma, leukemia, liver cancer, lung cancer, lymphoma, melanoma, mesothelioma, oral cancer, ovarian cancer, pancreatic cancer, prostate cancer, skin cancer, squamous cell cancer, stomach cancer, testicular cancer, thyroid cancer, hepatoma, neuroendocrine cancer, liposarcoma, head and neck cancer and cholangiocarcinoma.

In a further embodiment, the present invention provides methods of inhibiting HRGP expression in a cell by administering an HRGP-antisense nucleotide molecule to the cell.

In yet a further embodiment, the present invention provides methods of inhibiting HRGP expression in a cell by using a ribozyme directed against an HRGP encoding mRNA molecule.

In yet another further embodiment, the present invention provides methods of inhibiting HRGP expression in a cell by using an antibody that specifically binds a protein that includes a thrombospondin-binding region of an HRGP molecule.

In another embodiment, the present invention provides a method of modulating the activity of transforming growth factor-beta (TGF-beta). TGF-beta is involved in the regulation of a host of biological processes, including inflammation. Modulating TGF-beta activity by methods of the present invention thereby also results in modulation of inflammatory responses.

In one embodiment, the present invention provides a method of stimulating angiogenesis in a tissue by increasing the expression of HRGP, or by stimulating the activity of HRGP. This method may be applied in the treatment of conditions, disorders or diseases that are treatable by enhancing angiogenesis in the affected tissue, organ or whole organism. An example of a disorder or disease treatable by the methods of the present invention is cardiovascular disease.

Methods of the present invention for stimulating angiogenesis in a tissue by increasing the expression of HRGP, or by stimulating the activity of HRGP are applicable to instances of cardiovascular disease involving a coronary blood vessel, and particularly to a blocked coronary blood vessel. Furthermore, the methods of the present invention may be applied to prevent restenosis of a coronary blood vessel after treatment to remove the blockage.

Alternatively, angiogenesis may be usefully enhanced by the above methods of the present invention for stimulating angiogenesis in an animal, particularly a mammal, with a healing wound or suffering a non-healing wound (as in certain non-healing diabetic wounds).

The invention further provides methods for detecting, identifying, and quantifying compounds which modulate the thrombospondin-binding activity of a protein comprising the thrombospondin-binding motif of HRGP. Also, the invention contemplates diagnostic methods and methods for developing a prognosis for assessment the susceptibility of the malignant or pre-cancerous condition to therapies of the present invention based on the individual abundance and/or relative abundance of HRGP and/or thrombospondin in a biological fluid or tissue sample.

Such assay methods for detecting, identifying, and quantifying compounds which modulate the thrombospondin-binding activity of a protein comprising the thrombospondin-binding motif of HRGP include ELISA, RIA, immunohistochemistry or immunoassays. These assays may be in vivo or in vitro assays. The assays may be used as single tests for diagnosis and detection of modulators of the thrombospondin-binding activity, for instance of a tissue sample of an individual. Alternatively, the assays may be used to screen a whole library of proteins. For instance, the assays may be used for large-scale high-throughput screens in drug discovery.

Also contemplated by the present invention are assays to detect, identify or to quantify nucleic acids encoding proteins comprising a TSP-1-binding region from HRGP or a homolog of HRGP or fragments thereof in mammalian cell samples, tissue samples or other biological samples from mammalian sources.

The assays of the present invention are also useful to assess the presence or determine the amount of these nucleic acids encoding proteins comprising a TSP-1-binding region from HRGP or a homolog of HRGP or fragments thereof present. The assays may then be used to assess the susceptibility of the mammalian cell or tissue or the intact mammal to treatment with a compound of the present invention that modulates the expression of HRGP from a native HRGP encoding gene, or of an HRGP fragment expressed from a recombinant vector in a mammalian cell.

In yet another embodiment, the present invention provides a method of activating tissue plasminogen activator protein (tPA). The method includes a step of contacting the tPA with an immobilized protein that includes a thrombospondin-binding motif of HRGP. Alternatively, the method includes a step of contacting the tPA with an immobilized protein that includes a thrombospondin-binding motif of HRGP and plasminogen.

Yet further provided by the present invention is a method of promoting angiogenesis in the tissues of a mammal by administering to the mammal an effective amount of a protein that includes the thrombospondin-binding motif of HRGP. Alternatively, the method of promoting angiogenesis in the tissues of a mammal may be by administering to the mammal an effective amount of a compound that specifically increases the expression of a protein that includes the thrombospondin-binding motif of HRGP. In a particular embodiment, the protein that includes the thrombospondin-binding motif of HRGP is HRGP itself, or a homolog or fragment of HRGP that binds to thrombospondin.

In yet a further embodiment, the present invention provides a method of inhibiting tumor proliferation in a mammal by administering to the mammal an effective amount of an inhibitor of the binding of the thrombospondin-binding motif of HRGP to TSP-1. In a particular embodiment the inhibitor of the binding of the thrombospondin-binding motif of HRGP to TSP-1 is an antibody that specifically binds HRGP, or a fragment of an antibody that specifically binds HRGP.

Alternatively, the present invention provides a method of inhibiting tumor proliferation in a mammal by administering to the mammal an effective amount of a compound that inhibits expression of a protein comprising the thrombospondin-binding motif of HRGP. In a particular embodiment the inhibitor of the binding of the thrombospondin-binding motif of HRGP to TSP-1 is ribozyme specific for HRGP MRNA.

### BRIEF DESCRIPTION OF THE FIGURES

FIG. 1. HRGP contains CLESH-1 homology (thrombospondin-binding) motifs. Amino acid sequence alignment of CD36/LIMPII TSP binding motifs with homologous sequences in HRGP. Results of pattern-based search (BEAUTY) using CD36 exon 5 coding region (CD36 aa 95-143) as query identified a split CLESH-1 motif in HRGP (SEQ ID NO.1: aa 443-517). Optimization of alignments using SIM, ALIGN, and LALIGN programs also identified additional repeating motifs (SEQ ID NO.2: shown, aa 173-231). Amino acids identical between HRGP and either CD36 or LIMPII are highlighted white on black. Bold residues and pattern symbols represent conservative substitutions according to the following groups: basic [KRH, (+)]; acidic [DE, (−)]; charged [KRH, DE, (@)]; aromatic [YFW,(@)]; aliphatic [AG, (α)]; short chain [GA, STP, (!)]; hydrophobic [AGP, IVL, FM, (Δ)]; polar/hydrophilic [ST, KRH, DNEQ, CWY, (±)], hydroxyl [STY], and nonpolar/branched [IVL]. GenBank™/EMBL accession numbers: huHRGP, P04196; huCD36, M24795; huLIMPII, D12676.

FIG. 2. Model: HRGP inhibits the anti-angiogenic effect of TSP-1. bFGF-induced angiogenesis (left) is inhibited by TSP-1 through the interaction of the TSP type I repeat with the CLESH-1 domain of the signaling receptor CD36 (center). HRGP, which also contains the CLESH-1 motif, binds TSP-1, inhibiting the interaction of TSP-1 with CD36 thereby inhibiting the anti-angiogenic effect of TSP-1 (right).

FIG. 3. HRGP abrogates the effect of endogenous TSP in the corneal angiogenesis assay. Pellets containing bFGF at various doses with and without HRGP (50 ng) were implanted into the corneas of wild type C57BL/6 (closed symbols) or CD 36 null mice (open symbols), and area of neovascularization was measured after five days. HRGP (--●--) augmented the dose-dependent effect of bFGF (—▪—) in the wild type mice (p=0.0004 by ANOVA). In CD36 null mice, there was an enhanced dose response to bFGF (—□—) (p=0.01), but no effect seen with the addition of HRGP (--◯--). Results represent mean±SE of>6 replicates.

### DETAILED DESCRIPTION OF THE INVENTION

The present inventors have discovered that the binding of HRGP to thrombospondin (TSP-1) is localized to the thrombospondin type I repeats, the same sequence motifs responsible for CD36 binding. The present inventors have also discovered that HRGP modulates the interaction of TSP-1 with CD36, specifically in regulating the anti-angiogenic activity of TSP-1.

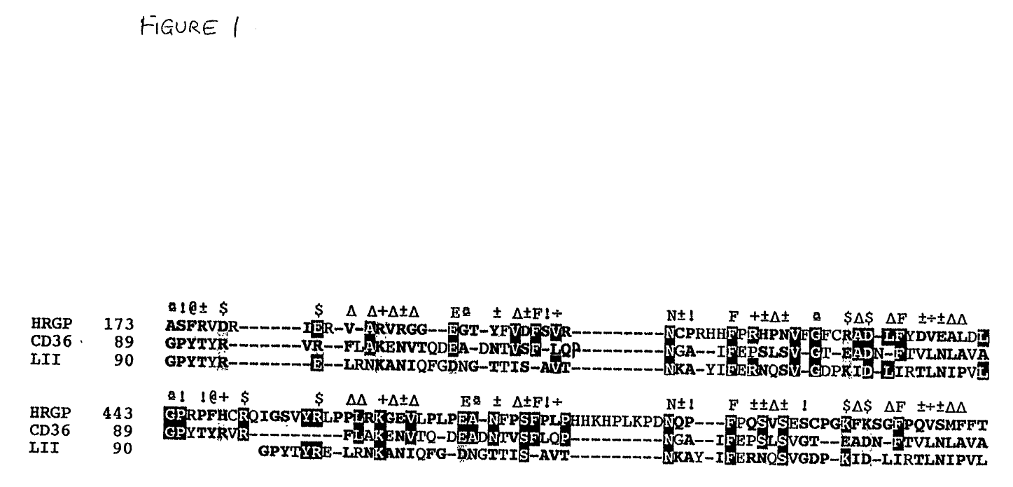

The thrombospondin-binding motifs are disclosed as amino acid sequences 443-517 and 173-231 of HRGP as shown in FIG. 1. The full amino acid sequence (1-525) of human HRGP is provided in GenBank under accession number P04196.

In one embodiment the invention relates to proteins comprising one or more thrombospondin-binding motifs from any animal, especially from a mammal, most preferably a human.

The protein may comprise HRGP, a fragment of HRGP or a fusion protein of HRGP or a fusion protein of a fragment of HRGP. The fragment or fusion protein of HRGP may have any molecular weight from about 7 Kda to about 60 Kda, or any molecular weight greater than about 70 Kda, but preferably less than about 200 Kda.

Alleles of HRGP and thrombospondin-binding motifs of HRGP as well as polymorphs and naturally occurring mutations, are encompassed by the present invention. The HRGP and proteins, conjugates and fusion molecules comprising one or more of the thrombospondin-binding motifs of HRGP may be in a native, naturally occurring form, chemically linked, or in a recombinant form, such as embodied in a fusion protein.

The present invention particularly concerns pharmaceutical compositions comprising proteins which are comprised of a thrombospondin-binding motif of HRGP or functional thrombospondin-binding fragments thereof, in a pharmaceutically acceptable carrier. The pharmaceutical composition of the present invention may be produced under GLP (Good Laboratory Practice) or GMP (Good Manufacturing Practice) conditions and is preferably of clinical grade. Particularly favored pharmaceutical compositions of the present invention are produced under GMP and are of clinical grade as required by the United States Food and Drug Administration, Center for Biologics Evaluation and Review (FDA, CBER).

The thrombospondin-binding motifs of the present invention also include functional fragments and homologs of the amino acid sequences 443-517 and 173-231 of HRGP (see FIG. 1) that retain the ability to bind TSP-1. The functional fragments may include combinations of sequences taken from the amino acid sequence 443-517 and from the amino acid sequence 173-231 of HRGP. Homologs of the amino acid sequences 443-517 and 173-231 of HRGP include sequence variants of each of these sequences, fragments of these variants and combinations of the fragments of these variants.

The amino acid sequence of a first protein is considered to be a homolog of a second sequence if the first amino acid sequence shares at least about 40% amino acid sequence identity, preferably at least about 50% identity, and more preferably at least about 70% identity, with the second sequence. In the case of proteins having high homology, the amino acid sequence of the first protein shares at least about 75% sequence identity, preferably at least about 85% identity, and more preferably at least about 95% identity, with the amino acid sequence of the second protein.

In order to compare a first amino acid or nucleic acid sequence to a second amino acid or nucleic acid sequence for the purpose of determining homology, the sequences are aligned so as to maximize the number of identical and conserved character (see next paragraph) amino acid residues or nucleotides. The sequences of highly homologous proteins and nucleic acid molecules can usually be aligned by visual inspection. If visual inspection is insufficient, the nucleic acid molecules may be aligned in accordance with the methods described by George, D. G. et al., in Macromolecular Sequencing and Synthesis, Selected Methods and Applications, pages 127-149, Alan R. Liss, Inc. (1988), such as formula 4 at page 137 using a match score of 1, a mismatch score of 0, and a gap penalty of −1.

The measure of homology as used herein is conservation of character of the amino acid residue of the HRGP or protein comprising a thrombospondin-binding motif. This can be, for example, a substitution, addition, or deletion mutant of the protein. For example, it is preferred to substitute amino acids in a sequence with equivalent amino acids. Groups of amino acids known normally to be equivalent are:

(a) Ala(A), Ser(S), Thr(T), Pro(P), Gly(G);

(b) Asn(N), Asp(D), Glu(E), Gln(Q);

(c) His(H), Arg(R), Lys(K);

(d) Met(M), Leu(L), Ile(I), Val(V); and

(e) Phe(F), Tyr(Y), Trp(W).

Substitutions, additions, and/or deletions in an amino acid sequence can be made as long as the protein encoded by the nucleic acid of the invention continues to satisfy the functional criteria described herein. An amino acid sequence that is substantially the same as another sequence, but that differs from the other sequence by means of one or more substitutions, additions, and/or deletions, is considered to be an equivalent sequence. Preferably, less than 60%, more preferably less than 40%, and still more preferably less than 20 or 30%, of the number of amino acid residues in a sequence are substituted for, added to, or deleted from the protein encoded by the nucleic acid of the invention.

Functional fragments of thrombospondin-binding motifs are defined as those portions of the amino acid sequences shown in FIG. 1 which retain thrombospondin binding activity. Such thrombospondin-binding fragments include, for example, regions 443-517 (SEQ ID NO. 1) and 173-231 (SEQ ID NO.2); also smaller fragments such as 443-451 (SEQ ID NO. 3); 452-480 (SEQ ID NO.4); 489-517 (SEQ ID NO.5); and also 173-179 (SEQ ID NO.6); 180-200 (SEQ ID NO.7); and 201-231 (SEQ ID NO.8).

The thrombospondin-binding sites may be comprised of any combination of the above-named fragments. The number of fragments may be any number from 2-10, for example

443-480 (SEQ ID NO.9) plus 452-480 (SEQ ID NO.4);

452-480 (SEQ ID NO.4) plus 489-517 (SEQ ID NO.5);

443-451 (SEQ ID NO.3) plus 489-517 (SEQ ID NO.5);

also other fragments, for example 173-200 (SEQ ID NO.10); and 180-231 (SEQ ID NO.11); may be used in combinations of sequences from the two regions, i.e. 443-517 (SEQ ID NO. 1) and 173-231 (SEQ ID NO.12) such as:

443-480 (SEQ ID NO.13) plus 180-231 (SEQ ID NO.11);

443-480 (SEQ ID NO.9) plus 201-231 (SEQ ID NO.8); and

180-231 (SEQ ID NO.11) plus 452-480 (SEQ ID NO.4) and so on.

Other combinations, including combinations sequences taken from any of the above recited SEQ ID NO.s will be immediately evident to those of skill in the art.

The present invention also particularly concerns methods of modulating the anti-angiogenic activity of thrombospondin (TSP-1) in the cell or target tissue of a mammal, particularly a human. Such modulation may be an inhibition of the anti-angiogenic activity of thrombospondin (TSP-1) or a stimulation of the anti-angiogenic activity of thrombospondin (TSP-1).

Inhibition of the anti-angiogenic activity of thrombospondin (TSP-1) may be achieved according to the methods of the present invention by stimulation of the expression of HRGP, or of a protein that includes a thrombospondin-binding motif of HRGP. Alternatively, the inhibition of the anti-angiogenic activity of thrombospondin (TSP-1) may be achieved according to the methods of the present invention by stimulation of the thrombospondin-binding activity of HRGP, or of a protein that includes a thrombospondin-binding motif of HRGP. These methods suppress the anti-angiogenic activity of TSP-1 by increasing the available TSP-1-binding activity in the cell or target tissue of a mammal and thereby inactivating the TSP-1 present in the cell.

Stimulation of the anti-angiogenic activity of thrombospondin (TSP-1) may be achieved according to the methods of the present invention by inhibition of the expression of HRGP, or of a protein that includes a thrombospondin-binding motif of HRGP. Alternatively, the stimulation of the anti-angiogenic activity of thrombospondin (TSP-1) may be achieved according to the methods of the present invention by inhibition of the thrombospondin-binding activity of HRGP, or of a protein that includes a thrombospondin-binding motif of HRGP.

The above method for inhibition of the thrombospondin-binding activity of HRGP includes for example, the use of anti-HRGP antibodies to inhibit the thrombospondin-binding activity of HRGP, or of a protein that includes a thrombospondin-binding motif of HRGP. Such inhibitors of HRGP expression or activity reduce the amount of free HRGP available to bind TSP-1 and thus enhance the anti-angiogenic activity of the TSP-1 present.

In one embodiment, the modulation is a stimulation of the activity of thrombospondin in a tissue comprising reducing the activity of HRGP. The reduction of activity of HRGP is accomplished by administering an inhibitor of HRGP activity or expression. The inhibitor of HRGP may also inhibit the activity or expression of a protein that includes a thrombospondin-binding motif, or CLESH-1 homologous sequence.

The inhibitor of expression of HRGP may be an HRGP-anti-sense molecule which blocks HRGP expression. U.S. Pat. No. 6,150,162 (the '162 patent) of Bennett and Cowsert discloses the antisense modulation of CD44 expression. These approaches to using antisense molecules for specific inhibition of expression of a protein are generally useful in the methods of the present invention. The entire specification of the '162 patent is hereby incorporated by reference. The review article of Gewirtz et al. (1998) Blood 92: (3) August 1, pages 712-736 entitled: “Nucleic Acid Therapeutics: State of the Art and Future Prospects” provides a useful summary of the currently available methods for using antisense nucleic acids as therapeutics.

The inhibitor of expression of HRGP may be an anti-HRGP ribozyme which blocks HRGP expression. U.S. Pat. No. 6,025,167 (the '167 patent) of Cech et al. discloses RNA Ribozyme polymerases, Dephosphorylases, Restriction Endoribonucleases and Methods. These approaches to using ribozyme molecules for specific inhibition of expression of a protein are generally useful in the methods of the present invention. The entire specification of the '167 patent is hereby incorporated by reference. The article of Bramlage et al. (1998) Trends Biotechnol Oct. 16: (10) pages 434-438 entitled: “Designing Ribozymes for the Inhibition of Gene Expression” provides a useful summary of the currently available ribozyme nucleic acid therapeutics.

In another preferred embodiment of the invention the inhibitor of activity of HRGP may be an antibody with binding specificity for HRGP, a single chain antibody with binding specificity for HRGP, or a fragment of any of the foregoing with binding specificity for HRGP.

In another embodiment, the stimulation of thrombospondin activity is particularly desirable where the tissue is a tumor undergoing angiogenesis. Such stimulation is achieved by inhibiting the activity of HRGP. An especially relevant example is where the tissue is a malignant tumor, especially a solid tumor. Some examples of solid tumors include breast, lung, prostate, colon, kidney, skin and other tumors.

Further the present invention also particularly concerns methods of inhibiting the activity of thrombospondin in a tissue comprising increasing the activity of HRGP. In a preferred embodiment the method of the present invention is applicable to any blood vessel including hepatic, carotid, femoral, brachial and all major arteries as well as capillaries and veins. The above method is particularly applicable to coronary blood vessels, and more especially blocked coronary blood vessels, such that angiogenesis (generation of new blood vessels in a tissue) is stimulated and blood supply to the blood and nutrient and oxygen-deprived tissue is restored. This method of the present invention for modulation of angiogenesis is especially useful to prevent restenosis. Stimulation of angiogenesis by the above described methods is also useful in stimulating healing of wounds, such as surgical wounds, and especially slow healing or non-healing wounds. Such wounds include are found for instance in the aged or in patients with diabetic conditions.

In particular embodiments the methods of the present invention may be used to modulate the activity of TGF-beta (transforming growth factor-beta) or tPA (tissue plasminogen activator). For example, the inventors have found that increasing HRGP expression leads to increased TGF-beta activity, which in turn enhances epithelial cell proliferation, growth and division and hence may be used to promote healing. Activation of tPA by methods herein described enhances anti-thrombotic activity, which may be a life-saving measure for patients suffering a heart attack due to a coronary or pulmonary embolism. Activation of tPA is achieved when either: HRGP protein or a protein comprising the thrombospondin-binding motif of HRGP is immobilized (such as in binding to a cell surface), or upon formation of a trimolecular complex comprised of an HRGP protein or protein comprising the thrombospondin-binding motif of HRGP with TSP-1, and plasminogen.

Assays, tests and screens for activation or inhibition of thrombospondin via inhibition or activation of HRGP respectively, are also provided by the present invention. Such inhibition of thrombospondin may be by inhibition of expression of TSP-1, e.g. by in vivo production of the HRGP or a thrombospondin-binding region of HRGP, or by inhibition of the thrombospondin-binding activity of an expressed HRGP protein or other molecule comprising a thrombospondin-binding motif of HRGP.

In one embodiment the invention provides a method for identifying compounds which modulate the thrombospondin-binding activity of a protein, comprising a thrombospondin-binding motif of HRGP.

The assay and screening methods of the present invention are particularly useful in identifying active molecules from small molecule libraries and libraries of biological molecules. Active molecules identified by these methods affect the binding of HRGP, or fragments or homologs with the thrombospondin molecule through the TSP-binding motif of the HRGP fragment or homolog of HRGP.

The molecules screened in the assays herein described are preferably small molecules or biological molecules. Biological molecules include all lipids and polymers of monosaccharides, amino acids and nucleotides having a molecular weight greater than 450. Thus, biological molecules include, for example, oligosaccharides and polysaccharides; oligopeptides, polypeptides, peptides, and proteins; and oligonucleotides and polynucleotides. Oligonucleotides and polynucleotides include, for example, DNA and RNA.

Biological molecules further include derivatives of any of the molecules described above. For example, derivatives of biological molecules include lipid and glycosylation derivatives of oligopeptides, polypeptides, peptides and proteins. Derivatives of biological molecules further include lipid and glycosylated derivatives of oligosaccharides and polysaccharides, e.g. lipopolysaccharides.

Any molecule that is not a biological molecule is considered in this specification to be a small molecule. Accordingly, small molecules include organic compounds, organometallic compounds, salts of organic and organometallic compounds, saccharides amino acids, and nucleotides. Small molecules further include molecules that would otherwise be considered biological molecules, except their molecular weight is not greater than 450. Thus, small molecules may be lipids, oligosaccharides, oligopeptides, and oligonucleotides, and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds.

In one embodiment the method for identifying a compound that specifically modulates the thrombospondin-binding activity in a cell may be recited as follows: First, the cell sample is contacted with a test compound; second, the thrombospondin-binding activity produced by the cell sample is assessed; and third, the thrombospondin-binding activity in the cell sample in the second step with the thrombospondin-binding activity produced by an identical cell which has not been contacted with the test compound are compared. Compounds identified by this assay as specifically modulating the thrombospondin-binding activity in a cell have no effect on cell samples lacking CD36 or on CD36 knockout mice. These mice are unaffected by changes in thrombospondin and thrombospondin binding activity, as they lack the target molecule, CD36 to which thrombospondin binds in order to cause thrombospondin mediated changes.

In another embodiment the method for identifying a compound that specifically modulates the thrombospondin-binding activity of a protein that carries a thrombospondin-binding motif of HRGP may be recited as follows: First, contacting a cell-free sample containing a protein that carries a thrombospondin-binding motif of HRGP, with a test compound and assessing the thrombospondin-binding activity in the cell free sample; and second, comparing the above-determined thrombospondin-binding activity in the cell-free sample with the thrombospondin-binding activity produced by an identical cell-free sample which has not been contacted with the test compound. Specific modulators of thrombospondin-binding activity of proteins having a thrombospondin-binding motif are recognized as those compounds identified as described above that have no effect in the absence of the thrombospondin target molecule, CD36.

The assessing of thrombospondin-binding activity in each of the steps of the above described assays may be detecting, in which presence or absence of thrombospondin-binding activity is assessed; alternatively, the assessing may be quantitative, in which case the thrombospondin-binding activity is assessed by amount, which may be numerically determined or comparatively assessed by reference to known or standard activity samples. The methods may also be used diagnostically to assess the susceptibility of samples, cells or tissues from a mammal to treatment with modulator of the present invention.

The determination of the amount of HRGP or thrombospondin-binding activity for diagnostic purposes may be assessed by any one of the following assay techniques: ELISA, RIA, immunochemistry or in vivo immunoassays.

In a preferred embodiment of the present invention the above assays may be applied to detect, assess or quantify the presence or absence of HRGP, or the level of HRGP activity (defined as thrombospondin-binding) in a biological fluid or tissue from a patient. The method may be used for assays in cell-free samples such as biological fluids (e.g. blood, plasma, lymph, saliva, sweat, tears, urine, and other such bodily fluids), and also for cell-based assays and tissue section assays as well as in vivo determinations.

In yet another preferred embodiment, thrombospondin activity from the above assay is compared with the thrombospondin activity in the cellular sample from the patient. Higher than average levels of HRGP are an indication of potential for malignancy due to suppression of the anti-angiogenic effect of thrombospondin. Even more serious is the situation where high levels of HRGP are accompanied by low levels or absence of thrombospondin. Patents in such cases have a poor prognosis without intervention and may be indicators for anti HRGP therapy or thrombospondin therapy. Unless treated such patients have a propensity for angiogenic changes and a higher than normal chance of developing tumors or other malignancies and should be considered for anti-HRGP treatment as herein described.

The present invention also provides a method of promoting angiogenesis in the tissues of a mammal, particularly a human, comprising administering an effective amount of a protein comprising the thrombospondin-binding motif of HRGP in a pharmaceutical carrier to the animal. Pharmaceutical carriers include neutral buffers such as Tris.HCl and phosphate buffers at about pH 7 with, or without added NaCl. Optionally the pharmaceutical carrier may also contain an inert material as a vehicle and may also contain a preservative, such as for example, an antioxidant.

The present invention further provides a method of inhibiting tumor proliferation in a mammal comprising administering to a mammal in need thereof an effective amount of an inhibitor (of the binding to TSP-1 of the thrombospondin-binding motif of HRGP) in a pharmaceutical carrier.

### USEFUL TECHNIQUES AND METHODS

PREPARATION OF PROTEIN

The protein and fragments of the present invention may be prepared by methods known in the art. Such methods include isolating the protein directly from cells, isolating or synthesizing DNA encoding the protein and using the DNA to produce recombinant protein, and synthesizing the protein chemically from individual amino acids.

Isolation of Protein from Solution

Proteins are isolated from the solubilized fraction by standard methods. Some suitable methods include precipitation and liquid/ chromatographic protocols such as ion exchange, hydrophobic interaction and gel filtration See, for example, Guide to Protein Chemistry Purification, Deutscher, M. P. (Ed.) Methods Enzymol., 182, Academic Press, Inc., New York (1990) and Scopes, R. K. and Cantor, C. R. (Eds.), Protein Purification (3d), Springer-Verlag, New York (1994).

Isolation of Protein from Gels

Alternatively, purified material is obtained by separating the protein on preparative SDS-PAGE gels, slicing out the band of interest and electroeluting the protein from the polyacrylamide matrix by methods known in the art. The detergent SDS is removed from the protein by known methods, such as by dialysis or the use of a suitable column, such as the Extracti-Gel column from Pierce.

Chemical Synthesis of Protein

The protein of the invention and DNA encoding the protein may also be chemically synthesized by methods known in the art. Suitable methods for synthesizing the protein are described by Stuart and Young in “Solid Phase Peptide Synthesis,” Second Edition, Pierce Chemical Company (1984), Solid Phase Peptide Synthesis, Methods Enzymol., 289, Academic Press, Inc, New York (1997). Suitable methods for synthesizing DNA are described by Caruthers in Science 230:281-285 (1985) and DNA Structure, Part A: Synthesis and Physical Analysis of DNA, Lilley, D. M. J. and Dahlberg, J. E. (Eds.), Methods Enzymol., 211, Academic Press, Inc., New York (1992).

RECOMBINANT PROTEIN

The protein may also be prepared by providing DNA that encodes the protein; amplifying or cloning the DNA in a suitable host; expressing the DNA in a suitable host; and harvesting the protein.

PROVIDING DNA

Chemical Synthesis from Nucleotides

The DNA may be synthesized chemically from the four nucleotides (A, T, G and C) in whole or in part by methods known in the art. Such methods include those described by Caruthers in Science 230:281-285 (1985) and DNA Structure, Part A: Synthesis and Physical Analysis of DNA, Lilley, D. M. J. and Dahlberg, J. E. (Eds.), Methods Enzymol., 211, Academic Press, Inc., New York (1992).

Alternatively, the nucleic acid molecules of the invention may be isolated from the available cDNA libraries and screened with selected probes designed to identify the gene of interest. See Sambrook, J. et al. (eds), Molecular Cloning, Second Edition, Cold Spring Harbor Laboratory Press, Cold Spring Harbor, New York (1989) and Ausubel, F. M. et al. (eds), Current Protocols in Molecular Biology, John Wiley & Sons, New York (1999).

DNA may also be synthesized by preparing overlapping double-stranded oligonucleotides, filling in the gaps, and ligating the ends together. The DNA may be cloned in a suitable host cell and expressed. The DNA and protein may be recovered from the host cell. See, generally, Sambrook, J. et al. (Eds.), Molecular Cloning, Second Edition, Cold Spring Harbor Laboratory Press, Cold Spring Harbor, New York (1989) and Ausubel, F. M. et al. (Eds.), Current Protocols in Molecular Biology, John Wiley & Sons, Inc., New York (1999).

Mutants obtained by Site-Directed Mutagenesis

The homlogs of the thrombospondin-binding motif of the present invention may be obtained by site directed mutagenesis. By this tehnique mutant DNA expressing the mutated protein may be prepared from wild-type DNA; see, for example, Zoller and Smith, Nucl. Acids Res. 10:6487-6500 (1982); Methods Enzymol. 100:468-500 (1983); DNA 3:479-488 (1984); Kunkel, T. A. et al., Methods Enzymol. 154:367-382, Academic Press, Inc., New York (1987); Uhlmann, E., Gene 71:29-40 (1988); Myers, R. M. et al., Science 229:242-246 (1985); Myers, R. M. et al., Methods Enzymol. 155, 501-527, Academic Press, Inc., New York (1987); and Current Protocols in Molecular Biology, Ausubel, F. M. et al. (Eds.), John Wiley & Sons, Inc., New York, (1999).

Expressing protein from DNA

The DNA encoding the protein of the invention may be replicated and used to express recombinant protein following insertion into a wide variety of host cells in a wide variety of cloning and expression vectors. The host may be prokaryotic or eukaryotic. The DNA may be obtained from natural sources and, optionally, modified. The genes may also be synthesized in whole or in part.

Cloning vectors may comprise segments of chromosomal, non-chromosomal and synthetic DNA sequences. Some suitable prokaryotic cloning vectors include plasmids from E. coli, such as colE1, pCR1, pBR322, pMB9, pUC, pKSM, and RP4. Prokaryotic vectors also include derivatives of phage DNA such as M13, fd, and other filamentous single-stranded DNA phages.

Vectors for expressing proteins such as the thrombospondin-binding proteins of the present invention in bacteria, especially E.coli, are also known. Such vectors include the pK233 (or any of the tac family of plasmids), T7, pBluescript II, bacteriophage lamba ZAP, and lambda PL (Wu, R. (Ed.), Recombinant DNA Methodology II, Methods Enzymol., Academic Press, Inc., New York, (1995)). Examples of vectors that express fusion proteins are PATH vectors described by Dieckmann and Tzagoloff in J. Biol. Chem. 260, 1513-1520 (1985). These vectors contain DNA sequences that encode anthranilate synthetase (TrpE) followed by a polylinker at the carboxy terminus. Other expression vector systems are based on beta-galactosidase (pEX); maltose binding protein (pMAL); glutathione S-transferase (pGST or PGEX) - see Smith, D. B. Methods Mol. Cell Biol. 4:220-229 (1993); Smith, D. B. and Johnson, K. S., Gene 67:31-40 (1988) ; and Peptide Res. 3:167 (1990), and TRX (thioredoxin) fusion protein (TRXFUS)—see LaVallie, R. et al., Bio/Technology 11:187-193 (1993).

Vectors useful for cloning and expression in yeast are available. Suitable examples are 2 μm circle plasmid, Ycp50, Yep24, Yrp7, Yip5, and pYAC3 (Ausubel, F. M. et al. (Eds.), Current Protocols in Molecular Biology, John Wiley & Sons, Inc., New York, (1999)).

Suitable cloning/expression vectors for use in mammalian cells are also known. Such vectors include well-known derivatives of SV-40, adenovirus, cytomegalovirus (CMV) retrovirus-derived DNA sequences. Any such vectors, when coupled with vectors derived from a combination of plasmids and phage DNA, i.e. shuttle vectors, allow for the isolation and identification of protein coding sequences in prokaryotes.

Further eukaryotic expression vectors are known in the art (e.g., P. J. Southern and P. Berg, J. Mol. Appl. Genet. 1:327-341 (1982); S. Subramani et al, Mol. Cell. Biol. 1:854-864 (1981); R. J. Kaufmann and P. A. Sharp, “Amplification And Expression Of Sequences Cotransfected with A Modular Dihydrofolate Reductase Complementary DNA Gene,” J. Mol. Biol. 159:601-621 (1982); R. J. Kautmann and P. A. Sharp, Mol. Cell. Biol. 159:601-664 (1982); S. I. Scahill et al, “Expression And Characterization Of The Product Of A Human Immune Interferon DNA Gene In Chinese Hamster Ovary Cells,” Proc. Natl. Acad. Sci. USA 80:4654-4659 (1983); G. Urlaub and L. A. Chasin, Proc. Natl. Acad. Sci. USA 77:4216-4220 (1980).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, the tet system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Useful expression hosts include well-known prokaryotic and eukaryotic cells. Some suitable prokaryotic hosts include, for example, E. coli, such as E. coli SG-936, E. coli HB 101, E. coli W3110, E. coli X1776, E. coli X2282, E. coli DH1, E. coli DH5αF′, and E. coli MRCl, Pseudomonas, Bacillus, such as Bacillus subtilis, and Streptomyces. Suitable eukaryotic cells include yeasts and other fungi, insect, animal cells, such as COS cells and CHO cells, human cells and plant cells in tissue culture.

Fusion Proteins

The proteins of the present invention may be expressed in the form of a fusion protein with an appropriate fusion partner. The fusion partner preferably facilitates purification and identification. Increased yields may be achieved when the fusion partner is expressed naturally in the host cell. Some useful fusion partners include beta-galactosidase (Gray, et al., Proc. Natl. Acad. Sci. USA 79:6598 (1982)); trpE (Itakura et al., Science 198:1056 (1977)); protein A (Uhlen et al., Gene 23:369 (1983)); glutathione S-transferase (Smith, D. B., Methods Mol. Cell Biol. 4:220-229 (1993); Smith, D. B. and Johnson, K. S. , Gene 67:31-40 (1988); Johnson, Nature 338:585 (1989)); Van Etten et al., Cell 58:669 (1989)); and maltose-binding protein (Guan et al., Gene 67:21-30 (1987); Maina et al., Gene 74:36-373 (1988), in Ausubel, F. M. et al. (Eds.) Current Protocols in Molecular Biology, John Wiley & Sons, Inc., New York (1999)).

Such fusion proteins may be purified by affinity chromatography using reagents that bind to the fusion partner. The reagent may be a specific ligand of the fusion partner or an antibody, preferably a monoclonal antibody. For example, fusion proteins containing beta-galactosidase may be purified by affinity chromatography using an anti-beta-galactosidase antibody column (Ullman, Gene. 29:27-31 (1984)). Similarly, fusion proteins containing maltose binding protein may be purified by affinity chromatography using a column containing cross-linked amylose; see Guan, European Patent Application 286,239.

The HRGP or HRGP fragment may occur at the amino-terminal or the carboxy-terminal side of the cleavage site. The fusion proteins of the present invention may also be His-tagged for ease of purification and isolation on metal charged affinity matrices. However, certain fusion proteins of regions of HRGP and certain HRGP fragments that have a high histidine content bind to metal charged affinity matrices without the need for inicorporation of a histidine tag.

Optionally, the DNA that encodes the fusion protein is engineered so that the fusion protein contains a cleavable site between the protein and the fusion partner. Both chemical and enzymatic cleavable sites are known in the art. Suitable examples of sites that are cleavable enzymatically include sites that are specifically recognized and cleaved by collagenase (Keil et al., FEBS Letters 56:292-296 (1975)); enterokinase (Hopp et al., Biotechnology 6, 1204-1210 (1988) Prickett, K. S. et al., Biotechniques 7:580-589 (1989); LaVallie et al., J. Biol. Chem. 268:23311-23317 (1993)); factor Xa (Nagai et al., Methods Enzymol. 153:461-481 (1987)); and thrombin (Eaton et al., Biochemistry 25:505 (1986) and Chang, J. Y. Eur. J. Biocehem. 151:217-224 (1985)). Collagenase cleaves between proline and X in the sequence Pro-X-Gly-Pro wherein X is a neutral amino acid. Enterokinase cleaves after lysine in the sequence Asp-Asp-Asp-Asp-Lys. Factor Xa cleaves after arginine in the sequence Ile-Glu or Asp-Gly-Arg. Thrombin cleaves between arginine and glycine in the sequence Arg-Gly-Ser-Pro.

Specific chemical cleavage agents are also known. For examples, cyanogen bromide cleaves at methionine residues in proteins (Gross, E., Methods Enzymol. 11:238-255 (1967), hydroxylamine cleaves at Asn-Gly bonds (Bornstein, G. and Balian, G., J. Biol. Chem. 245:4854-4856 (1970), and by hydrolysis at low pH (Asp-Pro bonds are labile at low pH; Landon, M., Methods Enzymol. 47(E):145-149 (1977).

ANTIBODIES

The present invention provides antibodies raised against a thrombospondin-binding protein of the present invention. An “antibody” in accordance with the present specification is defined broadly as a protein that binds specifically to an epitope or binding site. The antibody may be polyclonal or monoclonal. Antibodies further include recombinant polyclonal or monoclonal Fab fragments prepared in accordance with the method of Huse et al., Science 246, 1275-1281 (1989) and Coligan, J. E. et al. (Eds.) Current Protocols in Immunology, Wiley Intersciences, New York, (1999). The antibodies may be polyclonal or monoclonal.

Preparing Antibodies

Polyclonal antibodies are isolated from mammals that have been innoculated with the protein or a functional analog in accordance with methods known in the art (Coligan, J. E, et al. (Eds.), Current Protocols in Immunology, Wiley Intersciences, New York, (1999)). Briefly, polyclonal antibodies may be produced by injecting a host mammal, such as a rabbit, mouse, rat, or goat, with the protein or a fragment thereof capable of producing antibodies that distinguish between mutant and wild-type protein. The peptide or peptide fragment injected may contain the wild-type sequence or the mutant sequence. Sera from the mammal are extracted and screened to obtain polyclonal antibodies that are specific to the peptide or peptide fragment.

The antibodies are preferably monoclonal. Monoclonal antibodies may be produced by methods known in the art. These methods include the immunological method described by Kohler and Milstein in Nature 256:495-497 (1975) and by Campbell in “Monoclonal Antibody Technology, The Production and Characterization of Rodent and Human Hybridomas” in Burdon et al. (Eds.), Laboratory Techniques in Biochemistry and Molecular Biology, Volume 13, Elsevier Science Publishers, Amsterdam (1985); and Coligan, J. E, et al. (Eds.), Current Protocols in Immunology, Wiley Intersciences, New York, (1999); as well as the recombinant DNA method described by Huse et al., Science 246:1275-1281 (1989).

In order to produce monoclonal antibodies, a host mammal is inoculated with a peptide or peptide fragment as described above, and then boosted. Spleens are collected from inoculated mammals a few days after the final boost. Cell suspensions from the spleens are fused with a tumor cell in accordance with the general method described by Kohler and Milstein in Nature 256:495-497 (1975). See also Campbell, “Monoclonal Antibody Technology, The Production and Characterization of Rodent and Human Hybridomas” in Burdon et al. (Eds.), Laboratory Techniques in Biochemistry and Molecular Biology, Volume 13, Elsevier Science Publishers, Amsterdam (1985) and Coligan, J. E., et al. (Eds.), Current Protocols in Immunology, Wiley Intersciences, New York, (1999)).

Antibodies of the present invention include functional fragments that bind specifically to HRGP or to TSP-1. The fragments of the antibody may contain one or more complementarity determining region (CDR), and preferably contain all six CDRs of the whole antibody, although fragments containing fewer than all of such regions, such as three, four or five CDRs, may also be useful. Fragments may be prepared by methods described by Lamoyi et al in the Journal of Immunological Methods 56, 235-243 (1983) Lamoyi, E., Methods Enzymol. 121:652-663 (1986) and by Parham, P., J. Immunol. 131, 2895-2902 (1983). Specific Fab fragments can also be generated by using combinatorial phage display library as described in Clayton, R. et al., Biol. Reprod. 59:1180-1186 (1998) and in O'Brien, P. M. et al., Proc. Natl. Acad. Sci. USA 96:640-645 (1999).

Antibodies may also be prepared by screening a phage display library; obtaining phage nucleic acid encoding a protein with a functional binding region that binds to an antigen or epitope of interest; engineering the region of the phage nucleic acid that encodes the functional binding region into an immunoglobulin heavy chain or light chain or into a single chain antibody, or a functional fragment of any of the foregoing. For example, U.S. Pat. No. 5,977,322 of Marks & Schier, Regents of the Univ. of California. Humanized antibodies are preferred for treatment of patients as these molecules avoid the problems associated with immune reactions to foreign antigens. Humanized antibodies of the present invention may be prepared by methods well known in the art. For example see U.S. Pat. No. 5,859,205 hereby incorporated by reference in its entirety.

In order to be useful as an antigen, a peptide fragment must contain sufficient amino acid residues to define the epitope of the molecule being detected. If the fragment is too short to be immunogenic, it may be conjugated to a carrier molecule. Some suitable carrier molecules include keyhole limpet hemocyanin and bovine serum albumen. Conjugation may be carried out by methods known in the art (Coligan, J. E. et al. (Eds.) Current Protocols in Immunology, Chapter 9, Wiley Intersciences, New York, (1999)). One such method is to combine a cysteine residue of the fragment with a cysteine residue on the carrier molecule to form a bridging S-S bond between the fragment and the carrier molecule.

THERAPIES USING ANTI-HRGP ANTIBODIES

Anti-HRGP Antibodies and Proteins bearing TSP-1-Binding Motifs for Treatment

Animals, particularly mammals, including humans, suffering from a wide variety of disorders, imbalances and diseases are amenable to treatment by modulation of angiogenesis using HRGP-specific antibodies, proteins bearing TSP-1-binding motifs, or functional fragments of each as disclosed in the present invention.

Antibodies specific for HRGP or functional fragments thereof are useful in treatments for mammals where the suppression angiogenesis is indicated. For example, these HRGP-specific antibodies or functional fragments are useful in the treatment of any of a large number of disorders, conditions and diseases involving uncontrolled proliferation and differentiation commonly referred to as cancers.

Such disorders, conditions and diseases include anal cancer, bladder cancer, small cell lung cancer, non-small cell lung cancer, bone cancer, brain cancer, breast cancer, cervical cancer, chondrosarcoma, clear cell adenosarcoma (DES), colorectal cancer, endometrial cancer, esophageal cancer, cancer of the eye, cancer of the eyelid, kaposi's sarcoma, kidney cancer, cancer of the larynx, leiomyosarcoma, (soft tissue cancer), leukemia, liver cancer, lung cancer, lymphoma, melanoma, mesothelioma (asbestos), oral cancer, ovarian cancer, pancreatic cancer, prostate cancer, skin cancer, squamous cell cancer, stomach cancer, testicular cancer, thyroid cancer, hepatomas: (rare in U.S.), neuroendocrine cancer, liposarcoma, head and neck cancer and cholangiocarcinoma (intra-hepatic metastasis).

THERAPIES USING HRGP OR HRGP FRAGMENTS

HRGP or functional fragments thereof that specifically bind TSP-1 are useful in treatments for mammals where the stimulation of angiogenesis is indicated. For example, HRGP or functional fragments of HRGP are useful in the treatment of many disorders, conditions and diseases resulting from cardiovascular disease. Functional fragments of HRGP include those fragments that bind thrombospondin (TSP-1).

Bispecific antibodies, designed with dual antigenic specificities and prepared by chemically linking two different monoclonal antibodies or by fusing two hybridoma cell lines to produce a hybrid-hybridoma, are being developed as new agents for immunotherapy as described in Brennan, M. et al., Science 229:81-83 (1985); in Paulus, H., Behring Inst. Mitt. 78:118-132 (1985); in Rammensee, H. G. et al., Eur. J. Immunol. 17:433-436 (1987); in Segal, D. M. et al., Princess Takamatsu Symp. 19:323-331 (1988); in Kranz, D. M. et al., J. Hematother. 4:403-408 (1995); and in Morimoto, K. and Inouye, K. J. Immunol. Methods 224:43-50 (1999). Such antibodies may contain, for example, the F(ab′)₂ fragment or one or more Fab fragment.

NUCLEIC ACID MOLECULES

The present invention also includes isolated nucleic acid molecules that encode any of the HRGP proteins, HRGP fragments, HRGP homologs, TSP-1-binding proteins or the variable regions of antibodies that bind the TSP-1 type 1 region as described above. The nucleic acid molecule may be DNA or RNA.

PROBES

The present invention further provides a method of detecting the presence of thrombospondin-binding protein in a sample. The method involves use of a labelled probe such as an HRGP-binding protein or thrombospondin-binding protein or antibody that recognizes protein present in the sample which may be a fluid, particularly a biological fluid, an extract such as a tissue extract, a tissue or tissue section or other solid sample. The probe may be an antibody that recognizes protein or a fragment thereof.

Labelling of Probes

The probes described above are labelled in accordance with methods known in the art. The label may be a radioactive atom, an enzyme, or a chromophoric moiety. Methods for labelling antibodies have been described, for example, by Hunter and Greenwood in Nature 144:945 (1962) and by David et al. in Biochemistry 13:1014-1021 (1974). Additional methods for labelling antibodies have been described in U.S. Pat. Nos. 3,940,475 and 3,645,090 and in Harlow, E. and Lane, D., Using Antibodies, A Laboratory Manual, Cold Spring Harbor Laboratory Press, Cold Spring Harbor, N.Y. (1999).

The label may be radioactive. Some examples of useful radioactive labels include ³²P, ¹²⁵I, ¹³¹I, ³⁵S, ¹⁴C, and ³H. Use of radioactive labels have been described in U.K. 2,034,323, U.S. Pat. No. 4,358,535, and U.S. Pat. No. 4,302,204. Some examples of non-radioactive labels include enzymes, chromophores, atoms and molecules detectable by electron microscopy, and metal detectable by their magnetic properties.

Some useful enzymatic labels include enzymes that cause a detectable change in a substrate. Some useful enzymes and their substrates include, for example, horseradish peroxidase (pyrogallol and o-phenylenediamine), beta-galactosidase (fluorescein beta-D-galactopyranoside), and alkaline phosphatase (5-bromo-4-chloro-3-indolyl phosphate/nitro blue tetrazolium). The use of enzymatic labels have been described in U.K. 2,019,404, EP 63,879, in Ausubel, F. M. et al. (Eds.), Current Protocols in Molecular Biology, John Wiley & Sons, Inc., New York (1999), and by Rotman, Proc. Natl. Acad. Sci. USA 47:1981-1991 (1961).

Useful chromophores include, for example, fluorescent, chemiluminescent, and bioluminescent molecules, as well as dyes. Some specific chromophores useful in the present invention include, for example, luciferin, fluorescein, rhodamine, Texas red, phycoerythrin, umbelliferone, luminol and X-gal.

The labels may be conjugated to the antibody or other protein probe by methods that are well known in the art. The labels may be directly attached through a functional group on the probe. The probe either contains or can be caused to contain such a functional group. Some examples of suitable functional groups include, for example, amino, carboxyl, sulfhydryl, maleimide, isocyanate, isothiocyanate.

Alternatively, labels such as enzymes and chromophoric molecules may be conjugated to the antibodies or peptides or proteins by means of coupling agents, such as dialdehydes, carbodiimides, dimaleimides, and the like. The label may also be conjugated to the probe by means of a ligand attached to the probe by a method described above and a receptor for that ligand attached to the label. Any of the known ligand-receptor combinations is suitable. Some suitable ligand-receptor pairs include, for example, biotin-avidin or streptavidin, and antibody-antigen. The biotin-avidin combination is preferred.

Antibody Probes

The proteins and functional analogs of the invention may also be used to produce antibodies for use as probes to detect the presence of thrombospondin-binding proteins in a sample. The antibodies may be polyclonal or monoclonal.

Assays for detecting the presence of proteins with antibodies have been previously described, and follow known formats, such as standard blot and ELISA formats. These formats are normally based on incubating an antibody with a sample suspected of containing the protein and detecting the presence of a complex between the antibody and the protein. The antibody is labelled either before, during, or after the incubation step. The protein is preferably immobilized prior to detection. Immobilization may be accomplished by directly binding the protein to a solid surface, such as a microtiter well, or by binding the protein to immobilized antibodies.

In a preferred embodiment, a protein is immobilized on a solid support through an immobilized first antibody specific for the protein. The immobilized first antibody is incubated with a sample suspected of containing the protein. If present, the protein binds to the first antibody.

A second antibody, also specific for the protein, binds to the immobilized protein. The second antibody may be labelled by methods known in the art. Non-immobilized materials are washed away, and the presence of immobilized label indicates the presence of the protein. This and other immunoassays are described by David, et al. in U.S. Pat. No. 4,376,110 assigned to Hybritech, Inc., LaJolla, Calif.; by Coligan, J. E, et al. (Eds.), Current Protocols in Immunology, Wiley Intersciences, New York, 1999); and by Harlow, E. and Lane, D., Using Antibodies, A Laboratory Manual, Cold Spring Harbor Laboratory Press, Cold Spring Harbor, N.Y. (1999).

Immunoassays may involve one step or two steps. In a one-step assay, the target molecule, if it is present, is immobilized and incubated with a labelled antibody. The labelled antibody binds to the immobilized target molecule. After washing to remove unbound molecules, the sample is assayed for the presence of the label.

In a two-step assay, immobilized target molecule is incubated with an unlabelled first antibody. The target molecule-antibody complex, if present, is then bound to a second, labelled antibody that is specific for the unlabelled antibody. The sample is washed and assayed for the presence of the label, as described above. The immunometric assays described above include simultaneous sandwich, forward sandwich, and reverse sandwich immunoassays. These assays are well known to those skilled in the art.

In a forward sandwich immunoassay, a sample is first incubated with a solid phase immunoabsorbent containing antibody against the protein. Incubation is continued for a period of time sufficient to allow the protein in the sample to bind to the immobilized antibody in the solid phase. After the first incubation, the solid phase immunoabsorbent is separated from the incubation mixture and washed to remove excess protein and other interfering substances which also may be present in the sample. Solid phase immunoabsorbent-containing protein bound to the immobilized antibodies is subsequently incubated for a second time with soluble labeled antibody cross-reactive with a different domain on the protein. After the second incubation, another wash is performed to remove the unbound labeled antibody from the solid immunoabsorbent and to remove non-specifically bound labeled antibody. Labeled antibody bound to the solid phase immunoabsorbent is then detected and the amount of labeled antibody detected serves as a direct measure of the amount of antigen present in the original sample. Alternatively, labeled antibody that is not associated with the immunoabsorbent complex can also be detected, in which case the measure is in inverse proportion to the amount of antigen present in the sample. Forward sandwich assays are described, for example, in U.S. Pat. Nos. 3,867,517; 4,012,294; and 4,376,110.

In a reverse sandwich assay, the sample is initially incubated with labeled antibody. The solid phase immunoabsorbent containing immobilized antibody cross-reactive with a different domain on the protein is added the labeled antibody, and a second incubation is carried out. The initial washing step required by a forward sandwich assay is not required, although a wash is performed after the second incubation. Reverse sandwich assays have been described, for example, in U.S. Pat. Nos. 4,098,876 and 4,376,110.

In a simultaneous sandwich assay, the sample, the immunoabsorbent with immobilized antibody, and labeled soluble antibody specific to a different domain are incubated simultaneously in one incubation step. The simultaneous assay requires only a single incubation and does not require any washing steps. The use of a simultaneous assay is a very useful technique, providing ease of handling, homogeneity, reproducibility, linearity of the assays, and high precision. See U.S. Pat. No. 4,376,110 to David et al.

In each of the above assays, the sample containing antigen, solid phase immunoabsorbent with immobilized antibody and labeled soluble antibody are incubated under conditions and for a period of time sufficient to allow antigen to bind to the immobilized antibodies and to the soluble antibodies. In general, it is desirable to provide incubation conditions sufficient to bind as much antigen as possible, since this maximizes the binding of labeled antibody to the solid phase, thereby increasing the signal. The specific concentrations of labeled and immobilized antibodies, the temperature and time of incubation, as well as other such assay conditions, can be varied, depending upon various factors including the concentration of antigen in the sample, the nature of the sample an the like. Those skilled in the art will be able to determine operative and optimal assay conditions for each determination by employing routine experimentation.

There are many solid phase immunoabsorbents which have been employed and which can be used in the present invention. Well known immunoabsorbents include beads formed from glass, polystyrene, polypropylene, dextran, nylon, and other material; and tubes formed from or coated with such materials, and the like. The immobilized antibodies may be covalently or physically bound to the solid phase immunoabsorbent, by techniques such as covalent bonding via an amide or ester linkage or by absorption.

The following patents and scientific publications may be useful in practicing the full scope of the invention and are incorporated by reference in their entirety: U.S. Pat. No. 5,071,773 entitled “Hormone receptor-related bioassays” discloses assay methods using transcriptional reporter genes generally useful for high throughput screening. Such screens may be adapted for use of assays employing cell surface receptors and ion channels in addition to the steroid hormone receptors which act as transcription factors. U.S. Pat. No. 5,401,629 discloses further screening methods using readouts based on detecting changes in the transcription of reporter genes engineered to express a detectable signal in response to activation by intracellular signaling pathways.

### EXAMPLES

Materials and Methods

Reagents: Recombinant human basic fibroblast growth factor (bFGF) was purchased from R & D Systems Inc. (Minneapolis, Minn., USA) or from Research Diagnostics, Inc.(Flanders, N.J., USA). Rabbit antibody to HRGP was kindly supplied by Dr. Lawrence Leung, Stanford University, Palo Alto, Calif. Murine monoclonal antibody to TSP-1 (11.4) has been previously described (16). Murine monoclonal antibody to CD36 (FA6) was obtained from the Vth International Workshop on Human Leukocyte Antigens (17). TSP-1 was purified from human platelet releasate by heparin affinity and anion exchange chromatography on Mono Q-Sepharose (Pharnacia Biotech Inc., Piscataway, NJ, USA) as described (3, 16). Radiolabeling was performed with Na¹²⁵I (Amersham Life Science Inc., Arlington Heights, Ill., USA) using immobilized chloramine T (IODO-BEADS, Pierce, Rockford, Ill., USA) as described (18). Glutathione-S-transferase-CD36 fusion proteins (FP) have been previously described (19). HRGP was purified from human plasma by lys-plasminogen affinity chromatography as described (6). Purified proteins were incubated with polymyxin B-coated agarose (Sigma Chemical Co., St. Louis, Mo., USA) to remove any potentially contaminating lipopolysaccharides (LPS) prior to use in cellular assays. Specific rabbit antibody to CSVTCG was generated by subcutaneous immunization with KLH-coupled peptide. IgG was purified from serum by Protein A chromatography (Pierce).

Amino Acid Sequence Alignment: Sequence homology analysis was performed using blast enhanced alignment utility search (BEAUTY (20), SIN, ALIGN, and LALIGN (21) via search engines accessed through http://dot.imgen.bcm.tmc.edu.

Cell Culture: Human dermal microvascular EC (HMVEC) were purchased from Cascade Biologics, Inc. (Portland, Oreg., USA) and maintained in Medium 131 with 5% commercial Microvascular Growth Supplement (MVGS). CD36 expression, which was confirmed by flow cytometric analysis using a murine monoclonal anti-human CD36 antibody (FA6) and a fluorescein-conjugated goat anti-mouse IgG (Pharmingen, San Diego, Calif., USA), was maintained through passage 5.

Enzyme-linked immunosorbent assays (ELISA) were performed as described (6). Briefly, TSP-1 (4μg/ml) in coating buffer (0.5M sodium carbonate, pH 9.6) was applied to microtiter plates (Becton Dickinson, Lincoln Park, N.J., USA) for 3 hours at 37° C. After washing with wash buffer (0.05% Tween in 20 mM Tris, 150mM NaCl, pH 7.5), and blocking for 1 hour at 37° C. with 1% BSA in Tris-Tween, HRGP in varying amounts in the presence and absence of peptide was added for 18h at 4° C. After washing, alkaline phosphatase-conjugated anti-HRGP or nonimmune rabbit Fab'2 was added for 3h at 37° C., after which p-nitrophenyl phosphate substrate was added and optical density (OD) at 405 nm was read in a microplate reader (Molecular Devices, Menlo Park, Calif., USA).

Solid phase binding assays were performed as described (18). Briefly, HRGP in carbonate buffer was immobilized on 96-well strips (Immulon-4 Remove-a-well, Dynatech Laboratories Inc., Burlington, Mass., USA) overnight at 4° C. After blocking with 1% BSA, ¹²⁵I-TSP-1 was incubated in the presence of varying concentrations of CD36-FP in triplicate, and bound radioactivity was quantified on a gamma counter. Non-specific binding was determined by carrying out binding in the presence of excess unlabeled ligand.

Ligand Blot: TSP-1 was resolved on an SDS-polyacrylamide gel and electrophoretically blotted onto a polyvinylidene difluoride (PVDF) membrane. The membrane was blocked with 150 mM NaCl, 20 mM Tris (TBS) containing 5% BSA. Following three washes with TBS, washes the membranes were cut into strips and incubated with ligand(s) diluted in TBS, 0.1% BSA for 1 hour, washed, and developed by autoradiography.

Tube Formation Assay. Early passage (P2 or P3) HMVEC in 0.2% MVGS were grown on 150 μl of Matrigel (Becton Dickinson, Bedford, Mass., USA) at 3×10⁴ cells per well of 48-well plates. Reagents were added and cells were incubated at 5% CO₂, 37° C. for 24 hours then photographed. Images were scanned and number of branched structures was counted using Scion Image software (Frederick, Md., USA).

Endothelial Cell (EC) chemotaxis assays were performed as described using a modified Boyden chamber with 8 μm gelatin-coated membranes (22). Briefly, confluent flasks of HMVEC were grown in 0.1% MVGS overnight and plated at 3×10⁴ cells per well on a gelatinized porous membrane (Osmonics, Minnetonka, Minn., USA). Reagents were added to wells and cells were allowed to migrate at 5% CO₂, 37° C. for 4 hours. Membranes were stained and the number of cells migrating was counted. Results were expressed as the percentage of maximum cells migrating towards bFGF (38-113 cells/10 high power fields) minus the number of cells migrating in the absence of bFGF (4-13cells/10 hpf).

In vivo subcutaneous Matrigel plug assays were performed as described (23). Briefly, 500 μl of Matrigel mixed with proteins or growth factors was injected subcutaneously near the abdominal midline of C57B1/6 mice. Gels were removed after 10 days, fixed in 1% paraformaldehyde, embedded in paraffin, sectioned, and stained with hematoxylin and eosin. Immunohistochemistry was performed on unstained sections using an anti-human von Willebrand factor (vWF) IgG (Dako, Glostrup, Denmark) or isotype-matched control (Sigma) and a biotin-streptavidin-peroxidase antibody and development system (Vector Laboratories, Burlingame, Calif.) as described (24) and counterstained with Mayer's hematoxylin (ImmunoGenex, San Ramon, Calif., USA). After scanning, the degree of angiogenesis was determined by counts of vWF-positive blood vessels using Scion Image. For breast tissue, frozen sections of freshly obtained human breast carcinoma were incubated with rabbit antibody to HRGP or CSVTCG or murine monoclonal antibody to TSP, or isotype-matched controls (Sigma), and developed as above. These studies were approved by the Institutional Animal Use Committee.

In vivo corneal angiogenesis assays. Pellets composed of hydron (Hydro Med Sciences, Cranbury, N.J.) and sucralfate (Teva Pharmaceuticals, North Wales, Pa.) were mixed with proteins or growth factors, and were prepared and implanted into the comeas of C57B1/6 mice as described (38). After 5 days, eyes were viewed under a dissecting microscope and photographed with a Sony DKC-1000 digital camera. The area of neovascularization was calculated by measuring vessel length from limbus towards the pellet and number of clock hours of vascularization as described (39, 40). Studies were approved by the Institutional Animal Use Committee.

The type 1 repeat of TSP mediates binding to HRGP. HRGP or plasminogen binding to TSP-1 immobilized on plastic wells was measured by enzyme-linked immunosorbent assay (ELISA) as described above, using an alkaline phosphatase-conjugated anti-HRGP antibody and p-nitrophenyl phosphate substrate.

The binding of HRGP to TSP-1 was inhibited in the presence of 50 μM of the type-1 repeat synthetic peptide CSVTCG, but not by a scrambled peptide, TVSGCC or by an RGDS peptide. However, the binding of plasminogen to TSP-1 was not inhibited by the synthetic peptides. Ligand blots show binding of radiolabelled HRGP to TSP-1 that had been subjected to SDS-PAGE and transferred to nitrocellulose, then developed by autoradiography.

The type 1 repeat of TSP-1 mediates binding to HRGP. HRGP binds to TSP-1 saturably, reversibly, and with high affinity (7 nM) (6). Binding of HRGP to immobilized TSP-1 was inhibited by the TSP-1 hexapeptide CSVTCG, whereas the control TSP-1 peptide (RGDS) and the scrambled peptide (TVSGCC) had no effect. As an additional control, the binding of plasminogen to TSP-1 was measured. Plasminogen binding was not inhibited by the CSVTCG peptide (1B), further indicating that this interaction was not mediated by the type I repeats.

The TSP type 1 repeat inhibits the binding of HRGP to TSP-1 in a concentration-dependent manner. Varying amounts of HRGP were added alone or in the presence of increasing concentrations of the peptide CSVTCG to TSP-1 coated wells. Binding was measured by ELISA as described above. Inhibition of TSP-1 binding of HRGP by hexapeptide was concentration-dependent and reached maximum at 50 μM.

That the TSP-1-HRGP interaction is mediated by the TSP type I repeats is demonstrated by the following: Binding of radiolabelled HRGP to TSP-1 which had been resolved on SDS-PAGE and transferred to nitrocellulose was significantly decreased in the presence of anti-CSVTCG antiserum and completely abolished by the CSVTCG peptide (50 μM). Inhibition by the TSP-1 type I repeat was concentration dependent and reached maximum at 50 μM.

TSP-1 and HRGP co-localize in vivo. In order to determine if there is in vivo evidence for TSP-1-HRGP interactions, we performed immunohistochemical studies on serial sections of human breast cancer specimens. TSP-1 is abundantly expressed in breast tissue, particularly in the stroma and the basement membrane associated with malignant ductal epithelium (25-27). We used a murine monoclonal antibody to TSP-1 and a rabbit polyclonal anti-HRGP antibody to determine the localization of TSP-1 and HRGP. Both TSP-1 and HRGP were detected in the stromal connective tissue. Adjacent epithelial cells expressed TSP-1, however HRGP was not detected intracellularly.

TSP-1 and HRGP co-localize in stroma of human breast carcinoma. Frozen sections of freshly obtained tumor were incubated with monoclonal antibody to TSP-1, polyclonal anti-HRGP, or antiserum to CSVTCG. Slides were developed with a peroxidase-conjugated avidin-biotin second antibody system and examined at 500× magnification. Panels taken from adjacent sections and showed stromal connective tissue bands staining with anti-TSP and anti-HRGP but not anti-CSVTCG. Tumor cell stained with anti-TSP and anti-CSVTCG but not with anti-HRGP.

In order to explore whether the TSP type 1 repeat is involved in TSP-1-HRGP interactions in the breast cancer stroma, we developed a specific antibody to the CSVTCG peptide. The antiserum was reactive to plasmodium falciparum circumsporozoite protein, known to contain the peptide, and to purified TSP-1, by Western blot. The CSVCTG epitope was detectable intracellularly in the breast cancer cells where TSP-1 was detectable but HRGP was absent. However, in the tumor stroma where HRGP co-localized with TSP-1, there was no detectable CSVCTG reactivity. This provides evidence that TSP-1 associates with HRGP in vivo, and that this interaction masks the type I epitope of TSP-1.

HRGP contains CLESH-1 homology motifs. The binding site for TSP-1 on CD36 and other proteins is defined by homologous, evolutionarily conserved amino acid motifs known as CLESH-1 (18, 28, 29). As shown in FIG. 1, by sequence alignments of deduced amino acid sequences of HRGP we identified a region (aa 443-517) with significant homology to the CLESH-1 domain of CD36 and the CD36-related protein LIMPII. (31% identity, 74% similarity). We also identified additional repeating motifs, including aa 173-231 (20% identity, 70% similarity). Human immunodeficiency virus type I (HIV-1) also contains a CLESH-1 motif and may be susceptible to binding/immobilization by CLESH-1 binding and modulation of any biological activity dependent on the availability of the HIV-1gp120 CLESH-1 motif.

TSP-1 binding to HRGP: competition with CD36. Solid phase assays were performed in which the binding of radiolabelled TSP-1 to HRGP was measured. ¹²⁵I-TSP-1 binding to HRGP was inhibited by increasing concentrations of CD36 FP93-120, a CD36-glutathione-S-transeferase (GST) fusion protein which has been shown to bind to TSP-1 (12, 19). Similar concentrations of GST alone or CD36 FP298-439, which do not bind TSP-1, had no effect.

HRGP abolishes the anti-angiogenic activity of TSP-1. The process of angiogenesis requires distinct cellular activities, including migration, proliferation, and differentiation of endothelial cells (EC) into capillaries. TSP-1 inhibits EC differentiation in response to multiple angiogenic stimuli (11). The anti-angiogenic activity of TSP-1 is mediated by interaction of the type I TSP repeat with CD36, its high-affinity receptor on microvascular EC (12). We have recently reported that CD36 knockout mice, which have a grossly normal phenotype (30), lacked an anti-angiogenic response to TSP-1 in a cornea angiogenesis assay, providing further evidence that the presence of CD36 is necessary for the anti-angiogenic activity of TSP-1 (13).

HRGP inhibits the anti-angiogenic effect of TSP-1 in vitro. Microvascular EC were grown on Matrigel under low serum concentrations (0.2%) in the presence of bFGF (2 ng/ml), conditions which are known to induce tube formation. (a) The addition of TSP-1 (2 nM) inhibited bFGF-induced tube formation by EC (b). The anti-angiogenic activity of TSP-1 was abolished by the addition of HRGP (15 nM), which restored the ability of the EC to form tubular structures. (c). HRGP did not directly induce tube formation.

HRGP inhibits the anti-angiogenic effect of TSP-1 in vivo. Matrigel mixed was injected subcutaneously into the midline of mice and resulting plugs were harvested after 10 days, fixed, and paraffin embedded, sectioned, stained with. hematoxylin and eosin, and photographed at 200× magnification. Representative images from 4 separate experiments show the following: (A) bFGF alone stimulated angiogenesis; (B) bFGF+TSP−1 inhibited angiogenesis; (C) bFGF+TSP−1+HRGP reversed the inhibition due to TSP−1; (D) HRGP alone had no effect on bFGF stimulated angiogenesis, showing that the HRGP effect is mediated through interaction with TSP-1. Slides were also immunohistochemically stained with anti-vWF antibody and examined at 400× magnification.

In a second type of in vivo experiment, hydron/sucralfate pellets containing bFGF, TSP-1, and/or HRGP were implanted in the corneas of C57B1/6 mice. After 5 days, vigorous outgrowth of blood vessels was seen in 13/13 eyes implanted with pellets containing bFGF (50 ng) and in only 2/11 eyes implanted with pellets containing both bFGF and TSP-1 (200 ng) In mice implanted with pellets containing bFGF, TSP-1, and HRGP (100 ng, HRGP:TSP-1 molar ratio 3:1), angiogenesis was seen in 10/10 eyes (p=0.02, chi square analysis), with area of neovascularization 82.5% of that seen with bFGF alone (Table I). Similar results were obtained when HRGP was added in a separate pellet from bFGF and TSP-1. HRGP alone did not induce angiogenesis.

TABLE 1
HRGP reverses the CD36-dependent anti-angiogenic activity of
TSP-1 in a corneal angiogenesis assay.

| | | Area of Neovascularization | |
|---|---|---|---|
| Mouse | Pellet | (mm²) ± SE | N |
| Wild type | BFGF | 2.35 ± 0.10 | 13 |
| | BFGF + TSP-1 | 0.51 ± 0.21 | 11 |
| | BFGF + TSP-1 + HRGP | 1.94 ± 0.15* | 10 |
| | HRGP | 0 ± 0 | 4 |
| | HRGP ± TSP-1 | 0.01 ± 0.009 | 4 |
| | BFGF ± HRGP | 2.02 ± 0.33 | 4 |
| CD36 −/− | BFGF | 2.10 ± 0.14 | 6 |
| | BFGF +TSP-1 | 1.92 ± 0.32 | 8 |
| | BFGF + TSP-1 + HRGP | 2.30 ± 0.19 | 6 |
| | HRGP | 0.02 ± 0.004 | 4 |
| | HRGP ± TSP-1 | 0.01 ± 0.006 | 4 |

*p = 0.008.

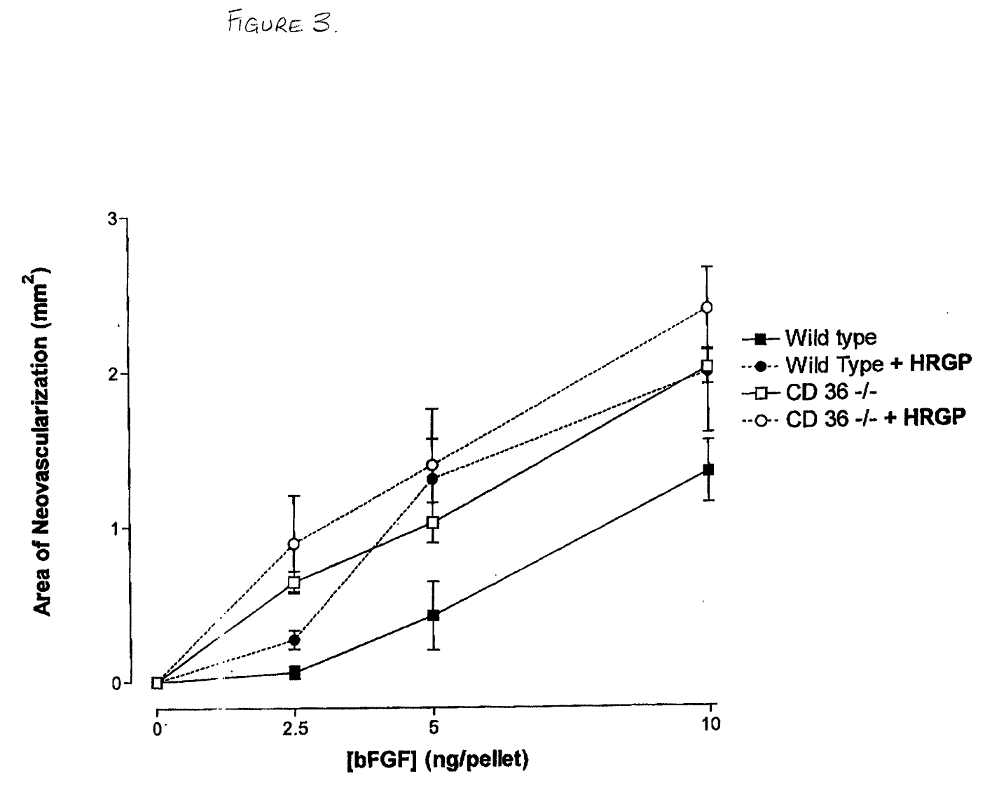

Although HRGP alone did not induce angiogenesis in corneal pocket assays, the angiogenic activity of bFGF was significantly increased in the presence of HRGP. As shown in FIG. 3, HRGP (50 ng) increased the potency of lower concentrations of bFGF, resulting in significantly larger areas of neovascularization at 2.5 to 10 ng of bFGF. Also shown in FIG. 3 is the result of experiments demonstrating that HRGP did not effect sensitivity to low concentrations of bFGF in CD36 null mice. In fact, the dose response curve of bFGF in wild type mice in the presence of HRGP was similar to that seen with bFGF alone in the CD36 null mice. TSP-1 did not inhibit bFGF-induced angiogenesis in the CD36 null mice, providing evidence that CD36 is necessary for the anti-angiogenic activity of TSP-1. These results were confirmed in an in vivo angiogenesis assay in which Matrigel impregnated with angiogenic growth factors was injected subcutaneously near the abdominal midline of mice. The resulting pellet was removed after 10 days, processed and stained for histologic examination and blood vessel counts. TSP-1 inhibited bFGF-induced blood vessel formation (14±8% of blood vessels/mm² seen with bFGF), and that HRGP inhibited the anti-angiogenic effect of TSP-1 (80±13%). These data demonstrate a physiologic role for HRGP in the modulation of the anti-angiogenic activity of TSP-1 in two distinct in vivo angiogenesis models.

HRGP inhibits the anti-chemotactic effect of TSP-1. Because the type I repeat also mediates the interaction of TSP-1 with HRGP, we studied the effect of HRGP on angiogenesis. EC migration towards bFGF was measured using a modified Boyden chamber with an 8 μm gelatin-coated membrane in the presence and absence of TSP-1 (2 nM) and increasing concentrations of HRGP (0-100 nM). Data from 3 separate experiments are expressed as a percentage of migration in the presence of bFGF alone. We measured migration of CD36-expressing microvascular EC towards bFGF. The addition of TSP-1 inhibited bFGF-induced migration of EC (approx. 30%±3% of control 100% value without added TSP-1). The anti-angiogenic activity was abolished by the addition of HRGP (approx. 75%±15% of control when 100 nM HRGP was added and approx. 30%±15% of control when 10 nM HRGP was added).

As a model for endothelial differentiation, we studied branched tube formation in Matrigel. bFGF induced tube formation by CD36-expressing microvascular EC grown on Matrigel under low serum concentrations. The addition of TSP-1 inhibited bFGF-induced tube formation, however, this was abolished by the addition of HRGP, which restored the ability of the EC to form tubular structures. HRGP alone, however, did not directly induce tube formation.

In vivo angiogenesis assays were performed in which Matrigel impregnated with angiogenic growth factors was injected subcutaneously near the abdominal midline of mice. The resulting pellet was removed after 10 days, processed and stained for histologic examination and blood vessel counts. We found that TSP-1 inhibited bFGF-induced blood vessel formation (14±8% of blood vessels/mm² seen with bFGF), and that HRGP inhibited the anti-angiogenic effect of TSP-1 (80±13%). These data suggest that HRGP specifically inhibits the anti-angiogenic activity of TSP-1 by interfering with the TSP-1-CD36 interaction.

### Discussion of Examples

The process of angiogenesis requires a complex set of interactions between endothelial cells and their surrounding matrix. Intricate control of the angiogenic process is necessary to allow for normal wound healing and development. In addition, tumor progression and metastasis is dependent on the supply of nutrients and growth factors by new blood vessels. Tumor masses which remain dormant and undetectable for years may suddenly acquire the ability to promote neovascularization, a process that has been termed angiogenic “switch” (31). Studies in murine models show that inhibition of angiogenesis may lead to regression of large tumor masses, suggesting that tumor angiogenesis is a dynamic process (32). Recent attention has focused on the identification and characterization of natural inhibitors of angiogenesis and their receptors, which provide attractive targets for therapeutic intervention.

Although several specific inhibitors of angiogenesis have been described, many of which have been found to be fragments of larger extracellular matrix-associated proteins (7, 33), TSP-1 is the only one for which a receptor-ligand interaction has been well-characterized. The chymotrypsin-resistant core of TSP-1, consisting of several properidin-like, type I repeats, retains the anti-angiogenic capacity of the intact molecule. Synthetic peptides derived from the type I domains have also been found to have potent anti-angiogenic activity in vivo and in assays of EC function. Although TSP-1 interacts with a number of proteins, its anti-angiogenic activity in vitro and in vivo is mediated by the glycoprotein receptor CD36 (12, 13). It was shown recently that inhibition of the TSP-1/CD36 interaction with a blocking antibody to TSP-1 leads to more rapid reendothelialization in a post-angioplasty setting, a process similar mechanistically to angiogenesis (34).

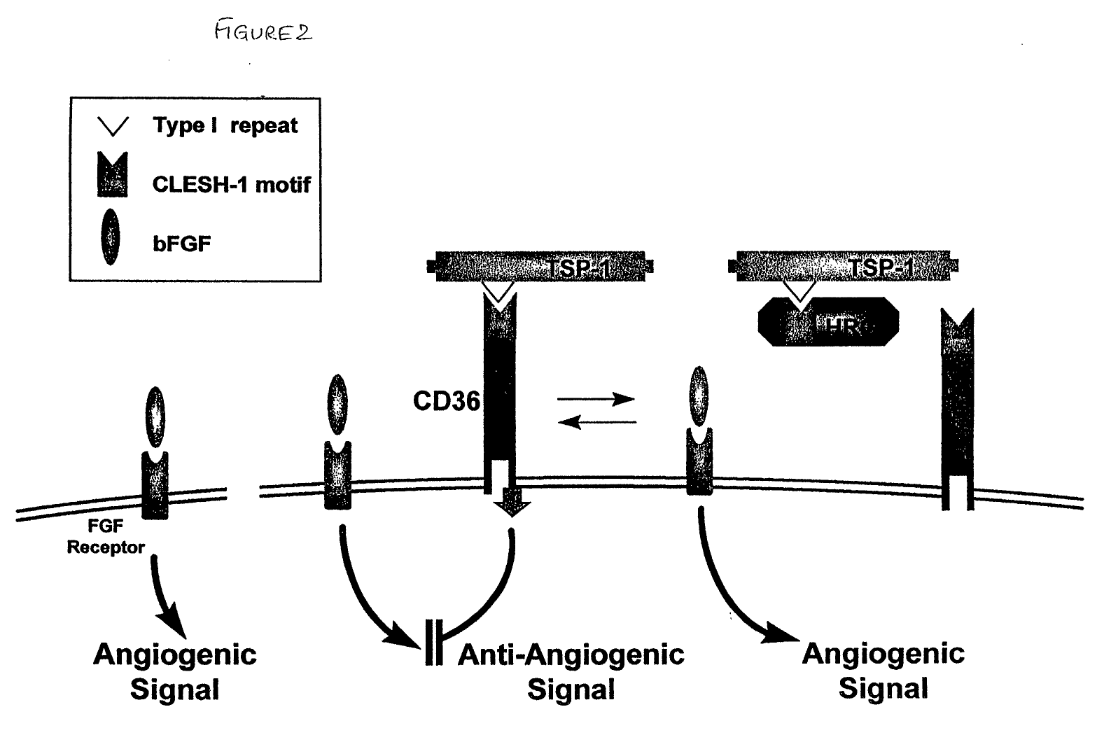

While a number of interactions have been reported for HRGP, no in vivo physiologic function has been described previously for this abundant plasma protein. The structure of HRGP is unique, consisting of a number of discrete domains including two cystatin-like domains at the amino terminus, a histidine rich region and two proline-rich domains (35). We have identified two regions in HRGP with significant homology to the TSP-1 binding site of CD36. These regions, known as CLESH-1 motifs, are conserved among members of the CD36 gene family and other TSP-1 binding proteins. Our studies show that binding of HRGP to TSP-1 was mediated by the TSP type I repeats, the same sequence motifs responsible for anti-angiogenic activity and CD36 binding. A model of the inhibition of the anti-angiogenic effect of TSP-1 by HRGP is herein disclosed (see FIG. 2). TSP-1 binds interaction of the type I repeats with the CLESH-1 domain of CD36, leading to a cascade of signaling events mediated by CD36. HRGP can interfere with the interaction of TSP-1 with CD36, and is an important natural modulator of angiogenesis.

HRGP is an important modulator of angiogenesis. Because the binding of HRGP to heparin is pH dependent and increases at lower pH, circulating HRGP may bind to glycosaminoglycans in matrix or on the surface of endothelial cells, especially in areas of relative hypoxia and low pH. HRGP in the matrix enhances angiogenesis by increasing fibrinolytic activity. Immobilized HRGP on a metal substrate (40) or in a trimolecular complex with TSP-1 and plasminogen (39) accelerates plasminogen, (PIg) activation by tPA. The rate of plasmin generation from the trimolecular complex was greater than from a bimolecular complex of TSP-Plg, indicating an important interaction of HRGP with Plg when both are complexed to TSP (39). The organization of a protein complex in the matrix may play an important role in the regulation of proteolytic activity. HRGP may serve as a natural, circulating mediator of the angiogenic switch.

Angiogenesis is involved in many forms of malignancy including tumors of lung, prostate gland, colon, kidney and skin, and is particularly relevant to the pathogenesis of breast cancer. The degree of angiogenesis in breast cancer specimens has been shown to correlate with the rate of metastasis, and the level of angiogenesis in breast cancers has been shown to be an independent prognostic factor, correlating with overall and relapse-free survival in patients with stage I and II carcinoma (36, 37). Primary human breast cancer cells express a number of proangiogenic factors such as bFGF and vascular endothelial growth factor, and TSP has long been known to be localized in the basement membrane and desmoplastic stroma of malignant breast epithelium (27). Using immunohistochemical studies of human breast cancer specimens, HRGP is shown to be co-localized with TSP-1 in the tumor matrix, and that this interaction masked the anti-angiogenic epitope of TSP-1. In areas where TSP-1 is an important inhibitor of angiogenesis, HRGP serves as a modulator of TSP-1 activity, promoting angiogenesis. The biologic impact of these interactions varies in different tumors according to the matrix components present.

Angiogenesis is critical for the growth and proliferation of tumors as well as for normal development. The present invention provides a novel role for histidine-rich glycoprotein (HRGP) in the modulation of angiogenesis. HRGP is a plasma protein which circulates in relatively high concentrations (1.5 μM), but heretofore had no known function in vivo. HRGP binds with high affinity to Thrombospondin-1, (TSP-1) a homotrimeric glycoprotein that is a potent inhibitor of angiogenesis. The anti-angiogenic activity of TSP-1 is mediated by the binding of properidin-like type I repeats to the receptor CD36.

Binding of HRGP to TSP-1 is similarly mediated by TSP type I repeats. HRGP is co-localized with TSP-1 in the stroma of human breast cancer specimens and this interaction masks the anti-angiogenic epitope of TSP-1. HRGP inhibits the anti-angiogenic effect of TSP-1 by interfering with the interaction of TSP-1 and CD36 in the following assays: in vitro assays of endothelial cell migration and tube formation, and in vivo Matrigel plug assays.

### References

1. Leung, L. 1993. Histidine-rich glycoprotein: an abundant plasma protein in search of a function. J Lab Clin Med. 121:630-1.

2. Leung, L. L. 1986. Interaction of histidine-rich glycoprotein with fibrinogen and fibrin. J Clin Invest. 77:1305-11.

3. Silverstein, R. L., Leung, L. L., Harpel, P. C., and Nachman, R. L. 1985. Platelet thrombospondin forms a trimolecular complex with plasminogen and histidine-rich glycoprotein. J Clin Invest. 75:2065-73.

4. Borza, D. B., and Morgan, W. T. 1997. Acceleration of plasminogen activation by tissue plasminogen activator on surface-bound histidine-proline-rich glycoprotein. J Biol Chem. 272:5718-26.

5. Angles-Cano, E., Gris, J. C ., Loyau, S., and Schved, J. F. 1993. Familial association of high levels of histidine-rich glycoprotein and plasminogen activator inhibitor-1 with venous thromboembolism. J Lab Clin Med. 121:646-53.

6. Leung, L. L., Nachman, R. L., and Harpel, P. C. 1984. Complex formation of platelet thrombospondin with histidine-rich glycoprotein. J Clin Invest. 73:5-12.

7. Good, D. J., Polverini, P. J., Rastinejad, F., Le Beau, M. M., Lemons, R. S., Frazier, W. A., and Bouck, N. P. 1990. A tumor suppressor-dependent inhibitor of angiogenesis is immunologically and functionally indistinguishable from a fragment of thrombospondin. Proc Natl Acad Sci USA. 87:6624-8.

8. Tolsma, S. S., Volpert, O. V., Good, D. J., Frazier, W. A., Polverini, P. J., and Bouck, N. 1993. Peptides derived from two separate domains of the matrix protein thrombospondin-l have anti-angiogenic activity. J Cell Biol. 122:497-511.

9. Bagavandoss, P., and Wilks, J. W. 1990. Specific inhibition of endothelial cell proliferation by thrombospondin. Biochein Biophys Res Commun. 170:867-72.

10. Frazier, W. A., Dixit, V. M., Galvin, N. J., and Rotwein, P. R. 1987. Structure of human thrombospondin: complete amino acid sequence derived from cDNA. Semin Thromb Hemost. 13:255-60.

11. Iruela-Arispe, M. L., Bornstein, P., and Sage, H. 1991. Thrombospondin exerts an antiangiogenic effect on cord formation by endothelial cells in vitro. Proc Natl Acad Sci USA. 88:5026-30.

12. Dawson, D. W., Pearce, S. F., Zhong, R., Silverstein, R. L., Frazier, W. A., and Bouck, N. P. 1997. CD36 mediates the In vitro inhibitory effects of thrombospondin-1 on endothelial cells. J Cell Biol. 138:707-17.

13. Jiminez, B., Volpert, O. V., Crawford, S. E., Febbraio, M., Silverstein, R. L., and Bouck, N. 1999. Signals leading to apoptosis-dependent inhibition of neovascularization by thrombospondin-1. Nature Medicine. in press.

14. Asch, A. S., Liu, I., Briccetti, F. M., Barnwell, J. W., Kwakye-Berko, F., Dokun, A., Goldberger, J., and Pernambuco, M. 1993. Analysis of CD36 binding domains: ligand specificity controlled by dephosphorylation of an ectodomain. Science. 262:1436-40.

15. Asch, A. S., Silbiger, S., Heimer, E., and Nachman, R. L. 1992. Thrombospondin sequence motif (CSVTCG) is responsible for CD36 binding. Biochem Biophys Res Commun. 182:1208-17.

16. Silverstein, R. L., and Nachman, R. L. 1987. Thrombospondin binds to monocytes-macrophages and mediates platelet- monocyte adhesion. J Clin Invest. 79:867-74.

17. Schlossman, S. F. 1995. Leucocyte typing V : white cell differentiation antigens: proceedings of the fifth international workshop and conference held in Boston, USA, 3-7 November, 1993. Oxford University Press, Oxford; New York. 2 v. (xxv, 2044, 31 )pp.

18. Crombie, R., Silverstein, R. L., MacLow, C., Pearce, S. F. A., Nachman, R. L., and Laurence, J. 1998. Identification of a CD36-related tbrombospondin 1-binding domain in HIV-1 envelope glycoprotein gpl20: relationship to HIV-1-specific inhibitory factors in human saliva. J Exp Med. 187:25-35.

19. Pearce, S. F. A., Wu, J., and Silverstein, R. L. 1995. Recombinant GST/CD36 fusion proteins define a thrombospondin binding domain. Evidence for a single calcium-dependent binding site on CD36. J Biol Chem. 270:2981-6.

20. Worley, K. C., Wiese, B. A., and Smith, R. F. 1995. BEAUTY: an enhanced BLAST-based search tool that integrates multiple biological information resources into sequence similarity search results. Genome Res. 5:173-84.

21. Huang, X. Q., Hardison, R. C., and Miller, W. 1990. A space-efficient algorithm for local similarities. Comput Appl Biosci. 6:373-81.

22. Polverini, P. J., Bouck, N. P., and Rastinejad, F. 1991. Assay and purification of naturally occurring inhibitor of angiogenesis. Methods Enzymol. 198:440-50.

23. Passaniti, A., Taylor, R. M., Pili, R., Guo, Y., Long, P. V., Haney, J. A., Pauly, R. R., Grant, D. S., and Martin, G. R. 1992. A simple, quantitative method for assessing angiogenesis and antiangiogenic agents using reconstituted basement membrane, heparin, and fibroblast growth factor. Lab Invest. 67:519-28.

24. Yamamoto, K., de Waard, V., Fearns, C., and Loskutoff, D. J. 1998. Tissue distribution and regulation of murine von Willebrand factor gene expression in vivo. Blood. 92:2791-801.

25. Tuszynski, G. P., and Nicosia, R. F. 1994. Localization of thrombospondin and its cysteine-serine-valine-threonine- cysteine-glycine-specific receptor in human breast carcinoma. Lab Invest. 70:228-33.

26. Clezardin, P., Frappart, L., Clerget, M., Pechoux, C., and Delmas, P. D. 1993. Expression of thrombospondin (TSP1) and its receptors (CD36 and CD51) in normal, hyperplastic, and neoplastic human breast. Cancer Res. 53:1421-30.

27. Wong, S. Y., Purdie, A. T., and Han, P. 1992. Thrombospondin and other possible related matrix proteins in malignant and benign breast disease. An inununohistochemical study. Am J Pathol. 140:1473-82.

28. Crombie, R., and Silverstein, R. 1998. Lysosomal integral membrane protein binds thrombospondin-1. Structure-function homology with the cell adhesion molecule CD36 defines a conserved recognition motif. J Biol Chem. 273:4855-63.

29. Pijuan-Thompson, V., Grammer, J. R., Stewart, J., Silverstein, R. L., Pearce, S. F., Tuszynski, G. P., Murphy-Ullrich, J. E., and Gladson, C. L. 1999. Retinoic acid alters the mechanism of attachment of malignant astrocytoma and neuroblastoma cells to thrombospondin- 1. Exp Cell Res. 249:86-101.

30. Febbraio, M., Abumrad, N. A., Hajjar, D. P., Sharma, K., Cheng, W., Pearce, S. F., and Silverstein, R. L. 1999. A null mutation in murine CD36 reveals an important role in fatty acid and lipoprotein metabolism. J Biol Chem. 274:19055-62.

31. Folkman, J., and Klagsbrun, M. 1987. Angiogenic factors. Science. 235:442-7.

32. O'Reilly, M. S., Holmgren, L., Chen, C., and Folkman, J. 1996. Angiostatin induces and sustains dormancy of human primary tumors in mice. Nat Med. 2:689-92.

33. O'Reilly, M. S., Holmgren, L., Shing, Y., Chen, C., Rosenthal, R. A., Moses, M., Lane, W. S., Cao, Y., Sage, E. H., and Folkman, J. 1994. Angiostatin: a novel angiogenesis inhibitor that mediates the suppression of metastases by a Lewis lung carcinoma. Cell. 79:315-28.

34. Chen, D., Asahara, T., Krasinski, K., Witzenbichler, B., Yang, J., Magner, M., Kearney, M., Frazier, W. A., Isner, J. M., and Andres, V. 1999. Antibody blockade of thrombospondin accelerates reendothelialization and reduces neointima formation in balloon-injured rat carotid artery. Circulation. 100:849-54.

35. Koide, T., and Odani, S. 1987. Histidine-rich glycoprotein is evolutionarily related to the cystatin superfamily. Presence of two cystatin domains in the N-terminal region. FEBS Lett. 216:17-21.

36. Weidner, N., Folkman, J., Pozza, F., Bevilacqua, P., Allred, E. N., Moore, D. H., Meli, S., and Gasparini, G. 1992. Tumor angiogenesis: a new significant and independent prognostic indicator in early-stage breast carcinoma [see comments]. J Natl Cancer Inst. 84:1875-87.

37. Weidner, N., Semple, J. P., Welch, W. R., and Folkman, J. 1991. Tumor angiogenesis and metastasis—correlation in invasive breast carcinoma. N Engl J Med. 324:1-8.

38. Volpert, O. V., Lawler, J., and Bouck, N. P. 1998. A human fibrosarcoma inhibits systemic angiogenesis and the growth of experimental metastases via thrombospondin-1. Proc Natl Acad Sci U S A. 95:6343-8.

39. Kenyon, B. M., Browne, F., and D'Amato, R. J. 1997. Effects of thalidomide and related metabolites in a mouse corneal model of neovascularization. Exp Eye Res. 64:971-8.

40. Rohan, R. M., Fernandez, A., Udagawa, T., Yuan, J., and D'Amato, R. J. 2000. Genetic heterogeneity of angiogenesis in mice. Faseb J. 14:871-6.

We claim:

1. A protein of molecular weight between about 7 Kd and about 60 Kd, or between about 70 Kd and about 200 Kd, wherein the protein comprises a thrombospondin-binding motif of HRGP.

2. A pharmaceutical composition comprising a protein which comprises a thrombospondin-binding motif of HRGP, in a pharmaceutically acceptable carrier.

3. The pharmaceutical composition according to claim 2, wherein the composition is produced under GMP conditions or is of clinical grade, or both.

4. A method of modulating the activity of thrombospondin in a tissue comprising: modulating the expression of a protein comprising a thrombospondin-binding motif of HRGP, or modulating the thrombospondin-binding activity of a protein comprising a thrombospondin-binding motif of HRGP.

5. The method according to claim 4, wherein the tissue is a human tissue.

6. The method according to claim 4, wherein the modulation of the activity of thrombospondin in the tissue is a stimulation of the activity of the thrombospondin comprising: inhibiting the expression of the protein comprising a thrombospondin-binding motif of HRGP, or inhibiting the thrombospondin-binding activity of the protein comprising a thrombospondin-binding motif of HRGP.

7. The method according to claim 6, wherein the tissue is a tumor undergoing angiogenesis.

8. The method according to claim 6, wherein the tissue has the characteristics of a cancer selected from the group consisting of: an anal cancer, a bladder cancer, a small cell lung cancer, a non-small cell lung cancer, a bone cancer, a brain cancer, a breast cancer, a cervical cancer, a chondrosarcoma, a clear cell adenosarcoma (DES), a colorectal cancer, an endometrial cancer, an esophageal cancer, a cancer of the eye, a cancer of the eyelid, a kaposi's sarcoma, a kidney cancer, a cancer of the larynx, a leiomyosarcoma, a leukemia, a liver cancer, a lung cancer, a lymphoma, a melanoma, a mesothelioma, an oral cancer, an ovarian cancer, a pancreatic cancer, a prostate cancer, a skin cancer, a squamous cell cancer, a stomach cancer, a testicular cancer, a thyroid cancer, a hepatoma, a neuroendocrine cancer, a liposarcoma, a head and neck cancer and a cholangiocarcinoma.

9. The method according to claim 6, wherein the inhibition of the expression of the protein comprising a thrombospondin-binding motif of HRGP is by an HRGP-antisense molecule.

10. The method according to claim 6, wherein the inhibition of the expression of the protein comprising a thrombospondin-binding motif of HRGP is by a ribozyme molecule.

11. The method according to claim 6, wherein the inhibition of HRGP activity is by an antibody with specificity for HRGP, or by an antibody fragment with binding specificity for HRGP.

12. The method according to claim 4 in which the activity of TGF-beta is modulated.

13. The method according to claim 4, wherein the modulation of the activity of thrombospondin in the tissue is an inhibition of the activity of the thrombospondin, comprising: stimulating the expression of HRGP or stimulating the thrombospondin-binding activity of HRGP.

14. The method according to claim 4, wherein the modulation of the activity of thrombospondin in the tissue is a inhibition of the activity of the thrombospondin comprising: inhibiting the activity of thrombospondin in a tissue, comprising increasing the expression of HRGP or increasing the thrombospondin-binding activity of HRGP.

15. The method according to claim 14, wherein the tissue comprises a blood vessel.

16. The method according to claim 15, wherein the blood vessel is a coronary blood vessel.

17. The method according to claim 16, wherein the coronary blood vessel is blocked.

18. The method of claim 16, wherein coronary blood vessel has been treated and restenosis of the blood vessel is prevented.

19. The method according to claim 14 wherein angiogenesis is stimulated.

20. The method according to claim 19 wherein the tissue is a wound tissue.

21. The method of claim 20 wherein the wound tissue is a non-healing diabetic wound tissue.

22. A method of identifying a compound that modulates the thrombospondin-binding activity in sample, said method comprising:
a) providing a sample that contains a protein with thrombospondin-binding activity,
b) contacting the sample with a test compound,
c) assessing the thrombospondin-binding activity of the sample, and
d) comparing the thrombospondin-binding activity produced by the sample in
a) with the thrombospondin-binding activity produced by an identical sample which has not been contacted with the test compound.

23. The method according to claim 22, wherein the sample is a cell sample.

24. The method according to claim 22, wherein the sample is a cell-free sample.
b) The method according claim 22, wherein the sample comprises a protein or peptide that comprises a thrombospondin-binding motif of HRGP.

25. The method of claim 22 in which the thrombospondin-binding activity is assessed by an assay technique selected from the group consisting of: an ELISA, an RIA, an immunohistochemical assay and an in vivo immunoassays.

26. A method of activating tissue plasminogen activator comprising:
a) contacting the tPA with an immobilized protein comprising a thrombospondin-binding motif of HRGP, or
b) contacting the tPA with a protein comprising a thrombospondin-binding motif of HRGP and plasminogen; and forming a trimolecular complex of the protein comprising the thrombospondin-binding motif of HRGP with TSP-1 and plasminogen.

27. A method of promoting angiogenesis in the tissues of a mammal comprising administering to the mammal an effective amount of either a) a protein comprising the thrombospondin-binding motif of HRGP, or b) a compound that specifically increases the expression of a protein comprising the thrombospondin-binding motif of HRGP.

28. The method according to claim 27, wherein the protein comprising the thrombospondin-binding motif of HRGP is HRGP.

29. A method of inhibiting tumor proliferation in a mammal comprising administering to the mammal an effective amount of either a) an inhibitor of the binding of the thrombospondin-binding motif of HRGP to TSP-1, or b) a compound that inhibits expression of a protein comprising the thrombospondin-binding motif of HRGP.

30. The method according to claim 29, wherein the inhibitor of the binding of the thrombospondin-binding motif of HRGP to TSP-1 is an antibody that specifically binds HRGP, or a fragment of an antibody that specifically binds HRGP.

31. The method according to claim 29, wherein the compound that inhibits expression of a protein comprising the thrombospondin-binding motif of HRGP is a ribozyme specific for HRGP MRNA.
